(19) United States
(12) Patent Application Publication
(10) Pub. No.: US 2004/0116410 A1
(43) Pub. Date: Jun. 17, 2004

(54) Diazepinones as antiviral agents

(76) Inventors: Hidetsura Cho, Osaka (JP); Rocco Dean Gogliotti, Pinckney, MI (US); Harriet Wall Hamilton, Chelsea, MI (US); Alexei Krasutsky, Skokie, IL (US); Takeshi Nakamura, Osaka (JP); Hiroki Tada, Osaka (JP); Peter Craig Weber, Canton, MI (US)

Correspondence Address: Charles W Ashbrook, Warner Lambert Company, 2800 Plymouth Road, Ann Arbor, MI, 48105, US

(21) Appl. No.: 10/344,924

(22) PCT Filed: Aug. 7, 2001
(86) PCT No.: PCT/US01/24731

(51) Int. Cl. A61K 31/551; C07D 487/02

(52) U.S. Cl. 514/220; 540/498; 540/497

(57) ABSTRACT

The invention is novel compounds of formula (I) which exhibit an improved therapeutic index and improved metabolic stability which are useful in the treatment and/or prevention of herpes viral infections. Novel intermediates useful in the synthesis of the final compounds are also part of the invention.

### BACKGROUND OF THE INVENTION

Herpes virus infection of mammalian cells results in disease states causing disfigurement, pain, and expense to the host. A variety of herpes viruses are known, which cause disease such as herpes simplex I and II, cytomeglia retinitis, chicken pox and shingles, Epstein-Barr syndrome, Kaposi's Sarcoma, as well as others which may not yet be defined. There is a pressing need for improved therapy for treating these diseases. Currently, exclusive of vaccines, treatment involves primarily nucleoside drugs such as acyclovir, which target thymidine kinase and suffer from development of resistance. Additionally, these drugs do not eradicate latent virus, and thus only treat symptoms of the disease.

In this invention we describe a series of therapeutically useful agents, which attack a different point in the viral life cycle as compared to nucleoside inhibitors of herpes virus replication, the transcription of herpes virus immediate early genes and protein expression. By virtue of this mechanism of action, they offer a therapy which is complementary to existing agents, and may offer the advantage of less resistance potential and suppression of, or re-activation from latency of the virus. The generic structure of these drugs is shown in Formula I below. The key feature of this structure is the seven-membered ring, which is appended to a two-ring system. The nature of these two rings may be varied. The seven-membered ring contains a nitrogen adjacent to the ring juncture, which may or may not be substituted. Additionally an amide functionality is incorporated into this seven-membered ring. The amide may be further derivatized to incorporate chemical moieties which function as prodrugs or are active in and of themselves. This diaza-seven-membered ring, particularly when substituted on nitrogen, is key to the essence of the invention.

U.S. Pat. No. 5,489,586 discloses compound of formula

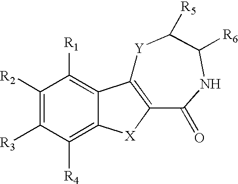

useful as agents which inhibit leukocyte adherence to vascular endothelium and as such are effective therapeutic agents for treating inflammatory diseases.

WO 96/29077 discloses compounds of formula

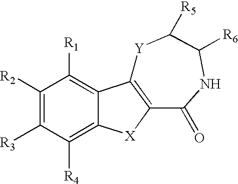

useful as therapeutic agents for treating viral diseases, including those caused by herpes virus and HIV.

The above two references are hereby incorporated by reference.

### SUMMARY OF THE INVENTION

The present invention relates to the extraordinary discovery that a substituted diazepine ring appropriately appended to a range of two-ring systems offers treatment of herpetic infections with an improved therapeutic index (TI) and improved metabolic stability. This encompasses compounds or pharmaceutically acceptable salts thereof, of Formula I

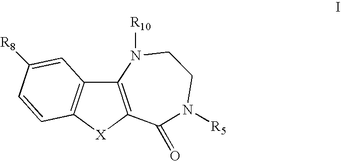

wherein R₈, R₁₀, R₅, and X are as described below.

Certain other novel compounds also exhibit these improvements.

The compounds are useful in the treatment and/or the prevention of herpes viral infections including conditions caused by herpes simplex I and II, herpes zoster, cytomegalovirus, Epstein-Barr virus, and VZV.

The compounds are useful in the treatment and/or the prevention of human herpes viruses 6, 7, and 8.

The invention is further a pharmaceutical composition of a compound of Formula I or the other compounds of the invention.

There are also novel intermediates useful in the preparation of the final products as part of the invention.

### DETAILED DESCRIPTION OF THE INVENTION

It was only when novel compounds were made in which nitrogen was the heteroatom in the seven-membered ring, and was substituted, combined with an appending two-ring system with appropriate substitution that the improved potency and TI were realized. Additionally, in vivo metabolism, found to be a liability with the compounds of the references was unexpectedly overcome. The compounds of this invention have improved metabolic stability. The invention lies in the unusual and unexpected combination of potency, therapeutic index, and metabolic stability conferred by the structures described below.

### Substitution on Nitrogen

Substitution on the nitrogen has been found to afford substantial increases in antiviral efficacy and significant improvement in TI; indeed the most potent compounds claimed are those with alkyl and alkyaryl (benzyl) substitutions. This was unexpected, because with the unsubstituted nitrogen (NH) compound no distinction in activity or TI was seen as compared to a sulfur or oxygen linkage. The chemical nature of the nitrogen atom allows three substituents, and therefor introduction of molecular mass into putative enzyme pockets which cannot be achieved with oxygen or sulfur as this part of the seven-membered ring. With these analogs we have realized a hundred-fold improvement in the concentration effective in killing 50% of the virus (EC₅₀). The concentration that kills 50% of the cell in the assay (toxic concentration, TC₅₀) has remained above micromolar levels, resulting in the improvement in TI. This is significant, and not previously known for any immediate early transcription inhibitors; neither was it foreseen in the anti-inflammatory structures.

### Other Substitutions on the Two-Fused Ring System Appended to the Diazepine Ring

In addition, based on the nitrogen containing seven-membered ring, we have been able to extend the nature of the two fused ring system beyond methoxy-benzothiophene to a variety of two-ring systems described below. As long as the key feature of the diazepine ring is present we have been able to append several heterocyclic and carbocyclic ring systems. These are exemplified by the naphthylene, isoquinoline, benzofuran, and indole systems, as well as the original benzothiophene. Substitution on the aromatic system has been extended to include a variety of functionalities which are more resistant to metabolic degradation.

The compound of the invention are compounds of Formula I

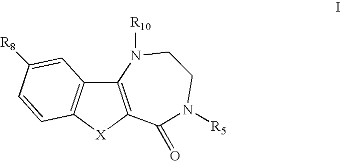

or a pharmaceutically acceptable salt thereof

wherein:

R₈ is H, F, Cl, Br, OR₁₁, NO₂, SO₂R₁₁, N(R₁₁)₂, CN, S—R₁₁ wherein R₁₁ is H, a straight or branched alkyl of 1-6 carbons having from 0 to 1 double or triple bonds, which alkyl is optionally substituted by 0 to 2 groups each independently selected from F, C₁, OR₁₂, and N(R₁₂)₂ wherein R₁₂ is H or straight or branched alkyl of from 1 to 4 carbons which alkyl is optionally substituted by F or OH;

R₁₀ is benzyl unsubstituted or substituted by alkyl, alkoxy, NO₂, halogen, tetrazole, or CN;

R₁₀ is also straight or branched alkyl of from 1 to 4 carbons substituted by 0 to 2 groups each independently selected from F, Cl, OR₁₂, and N(R₁₂)₂ wherein R₁₂ is as described above;

R₁₀ is also straight or branched alkyl of from 2 to 6 carbons having from 0 to 2 double bonds, which alkyl is unsubstituted or substituted;

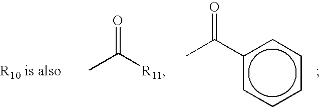

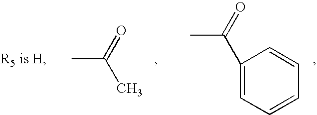

benzyl, or alkyl of from 1 to 4 carbons saturated or unsaturated; and

X is O or S.

The terms used in defining the compounds of the instant invention are defined below.

Alkyl is a straight or branched carbon chain of from 1 to 6 atoms and include, for example, methyl, ethyl, propyl, i-propyl, butyl, i-butyl pentyl, i-pentyl, or hexyl. The alkyl may be unsubstituted or substituted by one or more groups selected from alkyl, halogen, alkoxy, and nitro or as discussed in the invention.

Alkoxy is as defined for alkyl.

Alkenyl and alkynyl are as described for alkyl except one or more double or triple bonds occur.

Halogen is fluorine, chlorine, bromine, and iodine.

Benzyl is phenylmethyl and may be unsubstituted or substituted by alkyl, nitro, alkoxy, HO₂, halogen, tetrazole, or CN. The benzyl group is one key to the improved profile of compounds of the instant invention because it unexpectedly improved therapeutic index values of the compounds of this invention. The increase in therapeutic index was achieved by significantly improving the efficacy of the compounds without a parallel increase in toxicity; thus, a large increase in the ratio of the two (the definition of therapeutic index) as obtained. Comparison of compound B with compound C in Table 1 illustrates this point.

The compounds of the Formula I are capable of further forming pharmaceutically acceptable acid addition salts. All of these forms are within the scope of the present invention.

Pharmaceutically acceptable acid addition salts of the compounds of Formula I include salts derived from inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, N-methyl glutamine (see, for example, Berge S. M., et al., “Pharmaceutical Salts,” Journal of Pharmaceutical Science, 1977;66:1-19).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

For medical use, the amount required of a compound of Formula I or a pharmacologically acceptable acid addition salt thereof to achieve a therapeutic effect will, of course, vary both with the particular compound, the route of administration, the mammal under treatment, and the particular disorder of disease concerned. In a preferred embodiment, the invention provides a method for treating humans suffering from inflammatory disease, such as arthritis or swelling comprising administering an anti-inflammatory effective amount to the subject in need of treatment. A suitable dose of a compound of Formula I or a pharmacologically acceptable acid addition salt thereof for a mammal suffering from, or likely to suffer from any condition as described hereinbefore is 0.1 μg to 500 mg of the compound per kilogram body weight. In the case of systemic administration, the dose may be in the range of 0.5 to 500 mg of the compound per kilogram body weight, the most preferred dosage being 0.5 to 50 mg/kg of mammal body weight administered two to three times daily. In the case of topical administration, eg, to the skin or eye, a suitable dose may be in the range 0.1 ng to 100 μg of the compound per kilogram, typically about 0.1 μg/kg.

In the case of oral dosing for the treatment or prophylaxis of arthritis or inflammation in general, due to any cause, a suitable dose of a compound of Formula I or a physiologically acceptable acid addition salt thereof, may be as specified in the preceding paragraph, but most preferably is from 1 mg to 10 mg of the compound per kilogram, the most preferred dosage being from 1 mg to 5 mg/kg of mammal body weight, for example, from 1 to 2 mg/kg.

It is understood that the ordinarily skilled physician or veterinarian will readily determine and prescribe the effective amount of the compound to prevent or arrest the progress of the condition for which treatment is administered. In so proceeding, the physician or veterinarian could employ relatively low doses at first, subsequently increasing the dose until a maximum response is obtained.

While it is possible for an active ingredient to be administered alone, it is preferable to present it as a pharmaceutical formulation comprising a compound of Formula I or a pharmacologically acceptable acid addition salt thereof and a pharmacologically acceptable carrier therefor. Such formulations constitute a further feature of the present invention.

The formulations, both for veterinary and for human medical use, of the present invention comprise an active ingredient in association with a pharmaceutically acceptable carrier therefor and optionally other therapeutic ingredient(s). The carrier(s) must be ‘acceptable’ in the sense of being compatible with the other ingredients of the formulations and not deleterious to the recipient thereof.

The formulations include those in a form suitable for oral, pulmonary, ophthalmic, rectal, parenteral (including subcutaneous, intramuscular, and intravenous), intraarticular, topical, nasal, or buccal administration. Such formulations are understood to include long-acting formulations known in the art.

The formulations may conveniently be presented in unit dosage form and may be prepared by any of the methods well known in the art of pharmacy. All methods may include the step of bringing the active ingredient into association with the carrier which constitutes one or more accessory ingredients. In general, the formulations are prepared by uniformly and intimately bringing the active ingredient into association with a liquid carrier or a finely divided solid carrier or both, and then, if necessary, shaping the product into the desired formulation.

Formulations of the present invention suitable for oral administration may be in the form of discrete units such as capsules, cachets, tablets, or lozenges, each containing a predetermined amount of the active ingredient; in the form of a powder or granules; in the form of a solution or a suspension in an aqueous liquid or nonaqueous liquid; or in the form of an oil-in-water emulsion or a water-in-oil emulsion. The active ingredient may also be in the form of a bolus, electuary, or paste.

### Formulation for Oral Suspension

| Ingredient | Amount | |
|---|---|---|
| Compound of the Invention | 500 | mg |
| Sorbitol solution (70% N.F.) | 40 | mL |
| Sodium benzoate | 150 | mg |
| Saccharin | 10 | mg |
| Cherry Flavor | 50 | mg |
| Distilled Water q.s. adjusted | 100 | mL |

The sorbitol solution is added to 40 mL of distilled water and the oxazepinone is suspended thereon. The saccharin, sodium benzoate, and flavoring are added and dissolved. The volume is adjusted to 100 mL with distilled water. Each milliliter of syrup contains 5 mg of the oxazepinone. This oral formulation is ideally suited for treating inflammation in pediatric care.

### Preparation of Parenteral Solutions

In a solution of 700 mL of propylene glycol and 200 mL of distilled water for injection is dissolved 20.0 g of a compound of the investion. The pH of the solution is adjusted to 5.5 with hydrochloric acid, and the volume is made up to 1000 mL with distilled water. The formulation is sterilized, filled into 5.0 mL ampoules each containing 2.0 mL (representing 40 mg of active diazepinone) and sealed under nitrogen. The formulation is administered intravenously to patients suffering from a herpes viral infection.

### Preparation of Topical Cream

Five hundred milligrams of a compound of the invention is mixed with 15 g of cetyl alcohol, 1 g of sodium lauryl sulfate, 40 g of liquid silicone D.C. 200 (sold by Dow Corning Co., Midland, Mich.), 43 g of sterile water, 0.25 g of methylparaben, and 0.15 g of propylparaben. The mixture is warmed to about 75° C. with constant stirring, and then cooled to room temperature at which it congeals. The preparation is applied to the skin surface of a person suffering from herpes.

### Efficacy and Therapeutic Index of the Compounds for Inhibiting Herpes Virus Infections

The compounds of the present invention have been found to inhibit herpetic virus infections, and are therefore useful in the treatment of and prevention of herpes virus infections in mammals, especially herpes simplex I virus (HSV-1), as well as herpes simplex II virus (HSV-II), cytomegalovirus and varicella zoster virus. Herpesvirus constitutes a large group of DNA viruses found in many animal species. The nucleic acid is a single molecule of double-stranded DNA. The viruses mature in the nucleus of the infected cell, where they induce formation of a cytoplamic inclusion body. Herpesviruses are causative agents of conditions such as oral herpes simples, genital herpes simplex, varicella, herpes zoster, cytomegalic inclusio disease in humans, and of pseudorabies and other disease in animals.

Herpesviruses, using HSV-1 as the example, express genes in a temporal sense via transcriptional control. Three distinct groups of HSV-1 gene products are transcribed and translated in a coordinated fashion as a function of the viral life cycle. These groups are described as immediate early, early, or late genes or alternatively by α (alpha), β (beta), or γ (gamma) nomenclature. The immediate early genes, such as ICP4, are first transcribed by host transcription factors and host RNA polymerase II and are required for subsequent transcription of the early and late genes. Herpesvirus genes are generally transcribed from a single promoter for each gene and use cellular RNA II polymerase. The early genes are primarily required for viral DNA synthesis and the late genes of virion structural proteins. Transcription of the three classes of HSV-1 genes require host cellular transcription factors such as OTF-1 (octamer binding protein). Herpesviruses contain both cis acting DNA sequences and trans acting factors which work in concert with host transcription factors to regulate temporal gene expression. Characteristic of these viral transcription factors is α-TIF (immediate early trans-induction factor, VP16) which interacts with cellular nuclear factors such as OTF-1 and binds at a cis acting DNA sequence to trans activate the transcription of immediate early genes. Many of the same DNA sequence elements found in eukaryotic promoter such as TATA boxes, enhancer like elements, positive and negative regulators, and SPI binding sites are found in herpesvirus promoter sequences. As such, inhibition of viral transcription by interacting with host cellular proteins complexed with viral encoded transcription factors will prevent herpesvirus replication.

The compounds of the invention have exhibited excellent activity in standard assays utilized to measure anti-herpesvirus activity. For example, one assay utilized is called the “AVUS” screen.

This screen was designed to identify compounds which inhibit HSV-1 in phases of its life cycle from adsorption and penetration through late gene expression. The screen involves adding single compounds dissolved in MeOH at 20 mM to a monolayer of Vero cells to a final concentration of 25 μg/mL, then infecting the cells with a recombinant HSV-1, Us3::Tn5-lacZ. This virus contains an insertion of a lacZ gene driven by a viral late promoter in the US3 protein kinase gene of HSV-1. The infection is allowed to proceed for 20 hours, then the cells are lysed with a solution of Triton X-100 and CPRG in “Z” buffer and assayed for β-galactosidase activity. The positive control used is solvent alone without compound, which corresponds to 0% inhibition, and the negative control used is either no virus added to the wells or 0.5% Triton X-100 added to the wells, which corresponds to 100% inhibition. Percent inhibition of a compound is then calculated using these positive and negative controls. For selected compounds a titration of the compound is then assayed in both the β-galactosidase virus replication inhibition assay and a 2-day XTT toxicity assay in the absence of virus. This is used to determine the EC₅₀ and the TC₅₀, and thus the therapeutic index. Follow-up screens to this core set of AVUS screens include plagque reduction and yield reduction assays, with witld type HSV-I to verify antivral activity, and time course of addition studies to begin to dissect a possible mechanism of action.

The following Table 1 shows the anti-herpesvirus activity of compounds presented in this invention.

TABLE 1

| Example | | | Therapeutic |
|---|---|---|---|
| No. | EC₅₀ (μM) | TC₅₀ (μM) | Index |
| A | 0.125 | 7.6 | 61 |
| B | 0.103 | 4.5 | 59 |
| C | 0.0002 | 10 | 50000 |
| O | 0.015 | 18 | 1200 |
| K | 0.15 | 27% @ 6.25 μM | >42 |
| CC5 | 0.08 | 18.5 | 231 |
| Q | 0.08 | 6.0 | 75 |
| DD | 0.05 | 12.9 | 258 |
| Z | 0.33 | 16.1 | 49 |
| AA | 0.1 | 7.2 | 72 |
| EE2 | 0.029 | 3.4 | 117 |
| RR2 | 0.52 | 18% @ 50 | >96 |
| AA4 | 0.44 | 100 | 227 |
| GG4 | 0.35 | 11.5 | 33 |
| F | 0.055 | 1.7 | 31 |
| V | 0.1 | 1.3 | 22 |
| MM3 | 0.002 | 3 | 1500 |
| SS3 | 0.21 | 83 | 395 |
| WW3 | 0.032 | 10.2 | 319 |
| D | 0.08 | 100 | 1250 |
| W | 0.09 | 100 | 1111 |
| YY5 | 0.03 | 10 | 333 |
| TT | 0.002 | 0.4 | 200 |
| PP2 | 0.01 | 1.1 | 110 |
| U | <0.1 | 8.1 | >81 |
| Acyclovir | 1.0 | >100 | >100 |

### Metabolic Stability

Improved metabolic stability has been achieved with this type of antiviral structure using the features of this invention. The original sulfur substituted seven-membered ring was shown to have potential metabolic liability in an in vivo model to several different metabolites, with little parent drug detected These metabolites included de-methylation of the methoxy on the benzothiophene and oxidation of the sulfur of the seven-membered ring, among others not identified. Replacement of the sulfur with nitrogen has eliminated the possibility of this oxidation. Substitution of the seven-membered ring has imparted additional stability to metabolic breakdown, as measured by half-life in hepatic microsomes from mouse and human. In the case of the isoquinoline ring system no methoxy functionality is required, and in the case of the benzothiophene and benzofuran ring systems we are able to show replacement of the methoxy with a variety of functionality is possible while still maintaining efficacy as an antiviral agent. Thus the metabolic stability of the molecule was found to be markedly improved for the pharmaceutical use of these compounds.

The metabolic stability is measured as described, with Results shown in Table 2.

Compounds were individually incubated (30 μM, dissolved in DMSO) with human and mouse liver microsomes. The incubation consisted of 50 mM KPO₄, pH 7.4 buffer with 0.5 mg/mL pooled microsomal protein, 30 μM compound, and 1.0 mM NADPH. The total incubate volume was 0.5 mL. The incubate solution was pro-incubated for 3 minutes at 37° C. and the reaction initiated by the addition of NADPH. At 0, 10, 20, and 40 minutes 50 μL aliquots were removed and quenched with 100 μL acetonitrile and 25 μL of internal standard. Standard curves of each compound were prepared in a simialr manner at a concentration range of 7.5 μM to 45 μM. Th in vitro metabolic half-life determinations of these preparations were determined from the concentration vs. time plots using WinNonlin software. The in vitro half-life data represents the extent of oxidative and hydrolytic metabolism.

TABLE 2

| | t½ (min) Human Liver |
|---|---|
| Example No. | Microsome Preparation |
| A | 22 |
| B | 66 |
| F | >200 |
| H | 200 |
| K | 150 |
| FF4 | >200 |
| NN | 133 |

### Compound Preparation

The compounds required to practice the present invention may be prepared by the following methods. During the synthesis of some of the invention compounds, it may be necessary or desirable to convert reactive groups such as hydroxy, amino, and carboxy, to derivatives which will protect them from unwanted side reactions when a desired reaction is taking place somewhere else in the molecule. Such protected hydroxy, amino, and carboxy groups are readily deprotected by conventional methods. Commonly used chemical moieties which serve to protect reactive groups such as hydroxy, amino, and carboxy, and methods for their attachment and subsequent removal, are described by Greene and Wuts in Protective Groups in Organic Synthesis, John Wiley & Sons, Inc., New York, 1991. For example, an amino alkyl group (compound 6 in Schemes 1 and 2) can be reacted with benzylchloroformate, BOC-anhydride or the like, to form the protective carbamate. This can be removed at the desired time by appropriate reaction conditions (reduction, hydrolysis, etc.) to regenerate the free amino functionality.

The general methods for this scheme are described and examples given following the schemes.

### Formation of Diazepine Ring

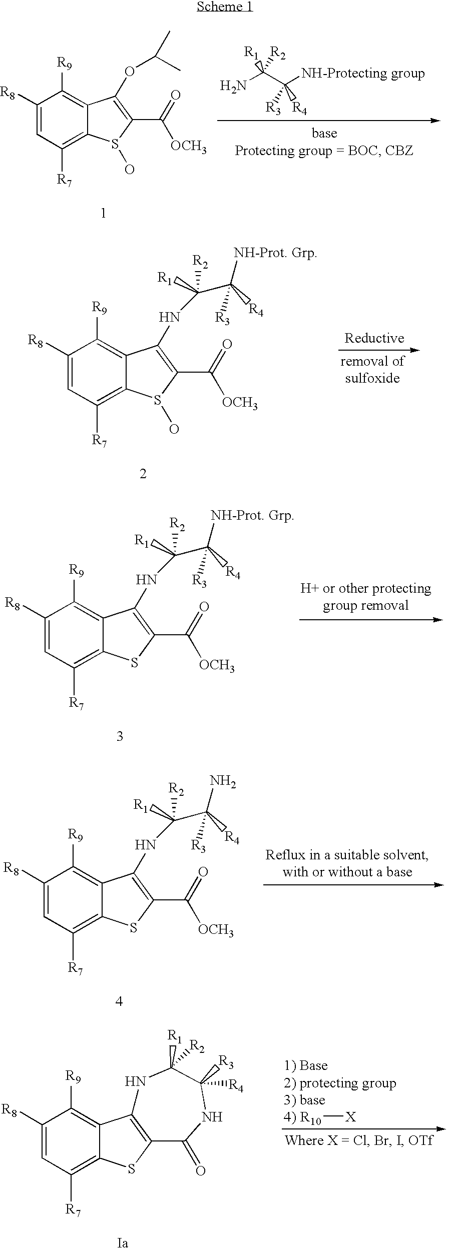

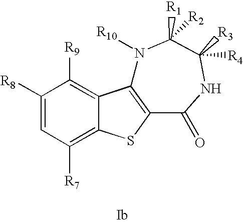

The first general approach requires as starting materials the benzothiophene sulfoxide carboxylate ester of structure 1 (Scheme 1), which is prepared as documented (J. Org. Chem. 1996;61:6060; J. Med. Chem. 1992;35:958). The conversion of compounds of type 1 to those of this invention is shown in Scheme 1. The benzothiophene sulfoxide carboxylate ester is treated with an amine of structure 6 such as 1-amino-2-alkyl-3-amino-butyloxycarbonyl in the presence of a base such as potassium t-butoxide or triethylamine if necessary, in tetrahydrofuran (TBF), acetonitrile, or other suitable solvent at 0 C to 80° C. to provide esters of type 2. The sulfoxide moiety is removed from the benzothiophene using reductive means such as catalytic hydrogenation or treatment with NaI/TMSCl in an appropriate solvent to afford a compound of structure 3. The protecting group on nitrogen in 3 is removed using a suitable acid such as HCl or trifluoroacetic acid in the case of the BOC group, to afford the corresponding primary amine 4. The preferred deprotection of the BOC group uses trifluoroacetic acid in dichloromethane, at a temperature of 0° C. to 25° C. In the case of the CBZ group, catalytic hydrogenation may be used, in a solvent such as THF or methanol, with a catalyst such as 20% palladium on carbon. The intermediate 4 is cyclized to Ia under basic conditions, preferably NaOMe in methanol, at a temperature of 25° C. to 80° C. Alternatively the amino ester 3 can be cyclized under acidic conditions such as polyphosphoric acid at elevated temperatures.

Compounds of structure 1a can be alkylated by protection of the carboxyamide using a suitable protecting group such as a substituted silyl group with is introduced under basic conditions, followed by alkylation. Thus treatment of Ia with a base such as sodium hydride or butyl lithium, followed by reaction with a chlorosilane such as t-butyl-dimethylsilyl chloride in an inert solvent such as tetrahydrofuran, at a temperature of 40° C. to 25° C. affords an intermediate protected compound which is not isolated. This intermediate is reacted with another equivalent of a suitable base such as butyl lithium, followed by the alkylating agent, which is generally (but not limited to) an alkyl or benzyl halide. This reaction may be done at a temperature of 40° C. to 80° C. After suitable workup, compounds of structure 1b are obtained.

An alternative route to the benzothiophene diazepines is shown in Scheme 2.

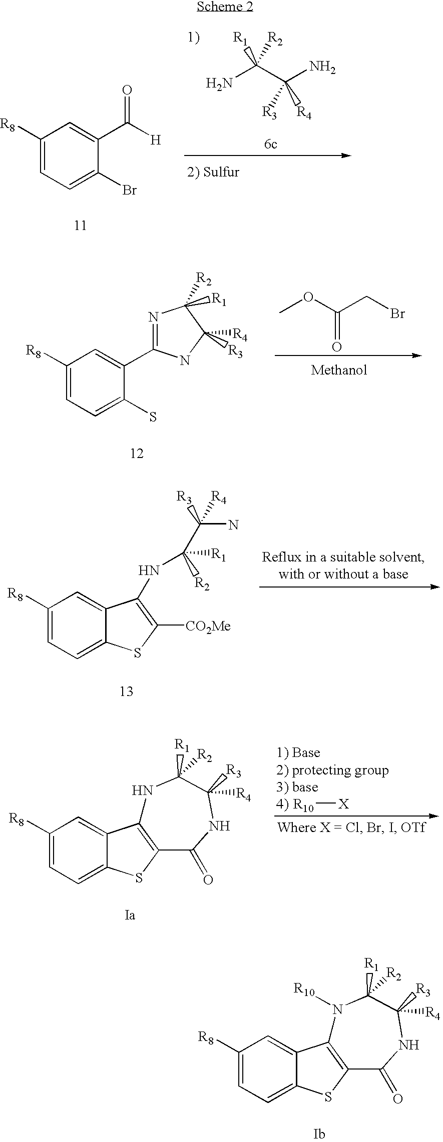

From the commercially available 11 a solution in 1-ethoxy ethanol or other suitable solvent is made, to which is added the diamine 6c. This is heated at an elevated temperature such as 100° C. for 1 to 10 hours, followed by slow addition of elemental sulfur (½ equivalent). The reaction is kept hot for another period of several hours (the solution turns black), and then cooled. The precipitate is collected, washed with water and dried to afford compound 12. This intermediate is dissolved in methanol or another protic solvent with methyl bromoacetate (1 equivalent) and refluxed for a period of several hours. After cooling the precipitate is filtered, and the filtrate concentrated. This residue is then recrystallized in an appropriate solvent such as chloroform. The crystals are collected and dried to yield 13. The final cyclization is conducted as described in Scheme 1, and the alkylation conducted in a similar manner if desired.

### Derivatization of R₈

If in the above schemes R₈ is methoxy, this can be functionalized as shown in Scheme 3.

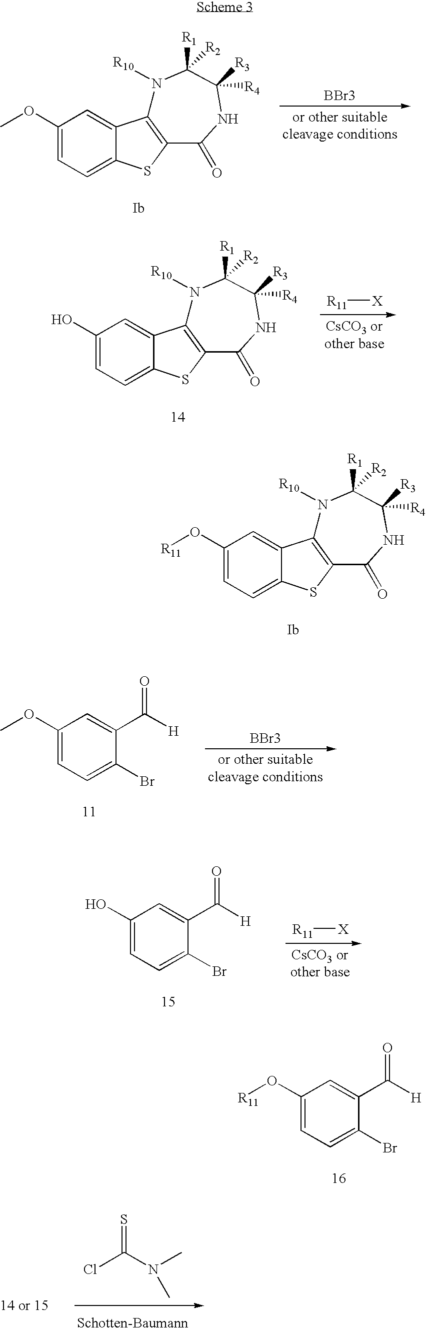

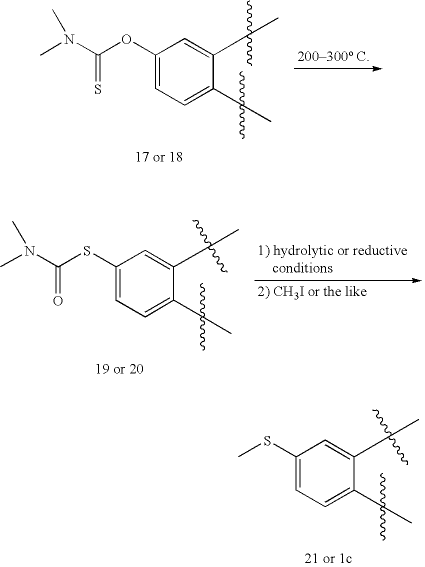

Treatment of the methoxy compound 1b with boron tribromide or a similar deprotection reagent to effect the removal of the methyl group and yield the free hydroxyl group is conducted, usually in a cooled chlorinated solvent such as methylene chloride. This is followed by work-up with 1N NaOH to free the complex and give the desired product. The hydroxyl can then be alkylated by known phenol alkylation conditions such as treatment with cesium carbonate and an alkyl halide, usually under reflux in an aprotic solvent. The reaction is cooled, filtered, and the filtrate concentrated then recrystallized in an appropriate solvent such as chloroform.

Alternatively, the same procedures can be followed on the starting methoxy compound 11 to give a compound which can then be used in Scheme 2 or carried on in Scheme 3.

From intermediate 14 or 15 in Scheme 3 other functionality can be achieved by reacting the compound under Schotten-Baumann conditions with ClCSNMe₂ to give the thiocarbamate 17 or 18. These compounds can be rearranged under elevated temperature (Newmann-Quart rearrangement) to afford 19 or 20, followed by treatment with strong base or reductive conditions to cleave off the amide and give the thiol. This thiol can then be alkylated with methyl iodide or another alkylating agent to gave the target compounds 21 or 1c.

Another approach to the functionalization of R₈ is shown in Scheme 4. This takes advantage of electrophilic aromatic chemistry to introduce various functional groups at this position.

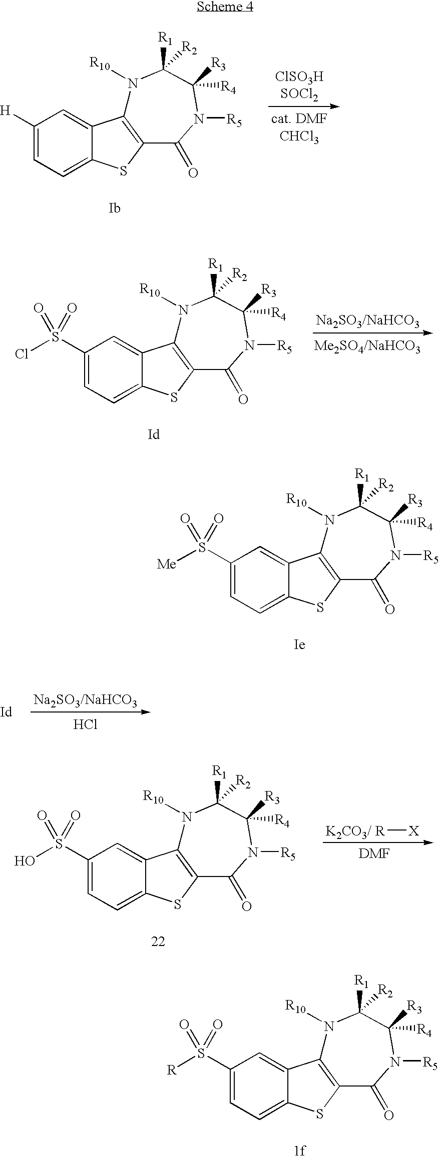

Thus the starting compound 1b where R₈ is H can be dissolved a mixture of chloroform and DMF and cooled, treated with ClSO₃H and thionyl chloride and the reaction heated at reflux as necessary. Material 1d is isolated crude for use in subsequent reactions.

Compound 1e is synthesized by heating an aqueous solution of Na₂SO₃ and sodium bicarbonate to 70-80° C. for 1 to 6 hours, then adding 1d. After further heating and isolation the intermediate is alkylated with Me₂SO₄ or another suitable alkylating agent and NaHCO₃ to give 1e.

Alternatively the intermediate (Na salt of sulfinate) can be prepared as above, the dissolved in H₂O and conc. HCl added to give the free acid. The solid 22 can be dissolved in DMF and treated with potassium carbonate and an alkylating agent such as ethyl iodide and the like and stirred for 1 to 8 hours at room temperature. The reaction can be worked up by partitioning between EtOAc and water and collecting the resulting solid, which is washed with water, and methanol, recrystallized from an appropriate solvent such as TBF to yield the final product 1f.

Other substitutions for the R₈ position can be introduced as outlined in Scheme 5.

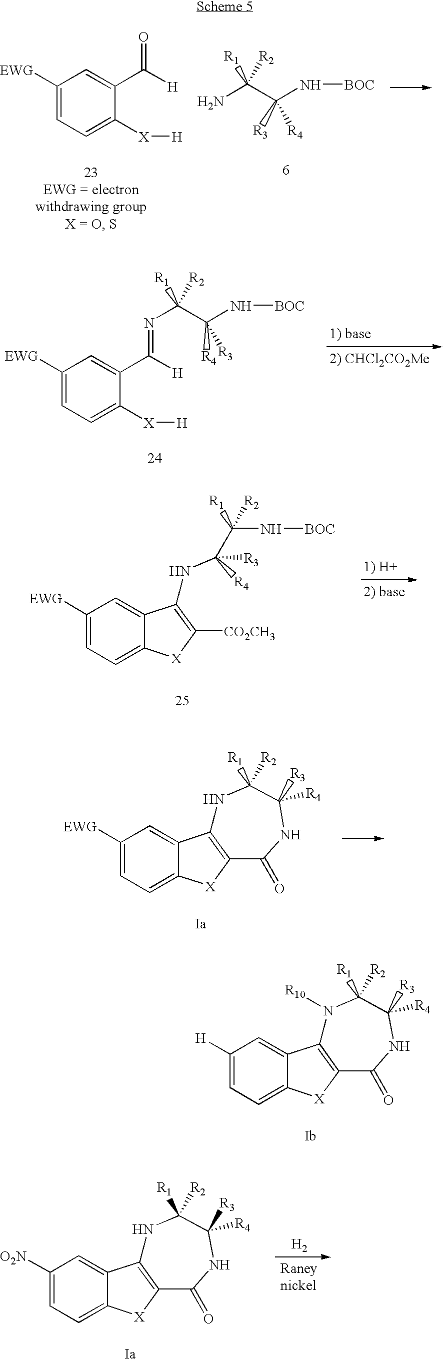

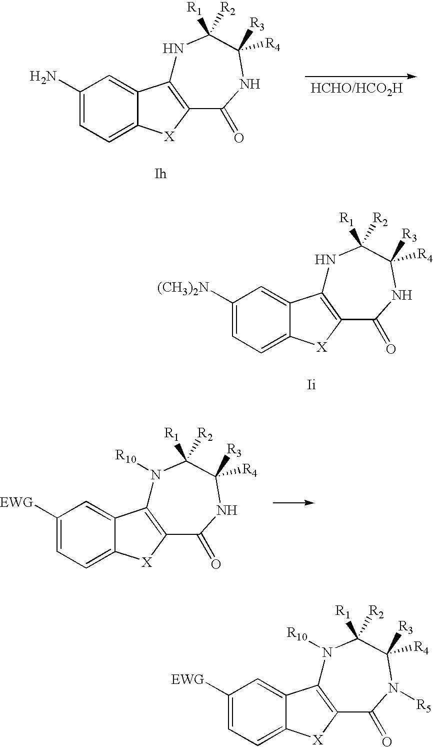

The route is similar to that of Scheme 2, with the particular provision that R₈ is an electron withdrawing group such as nitro, halogen, or the like, and that X can be either sulfur or oxygen.

In the scheme the aldehyde 23 is condensed with the mono-protected diamine 6 to give the imine 24, which is then treated with a base such as potassium carbonate and methyldichloroacetate in a suitable solvent such as DMF to effect ring closure to the benzothiophene or benzofuran 25. The BOC protecting group is removed under acidic conditions such as TFA or HCl in an appropriate solvent. The third ring is closed as previously described by treatment with base such as methoxide (if necessary) in an alcoholic solvent, usually with heat to give the target 1a.

Compound 1a can be further derivatized as outlined in Scheme 1 to compounds 1b.

Compound 1a from this scheme can also be reduced when EWG is nitro to afford the amino analog 1h, which can be reacted further, for example under Eschweiler-Clarke conditions to yield the alkylated amine 1i.

The compounds can also be derivatized as in Scheme 8 to substitute the amide nitrogen with an R₅ group.

Benzofuran compounds with electron donating groups such as methoxy for R₈ can also be synthesized in a similar manner as outlined in Scheme 6.

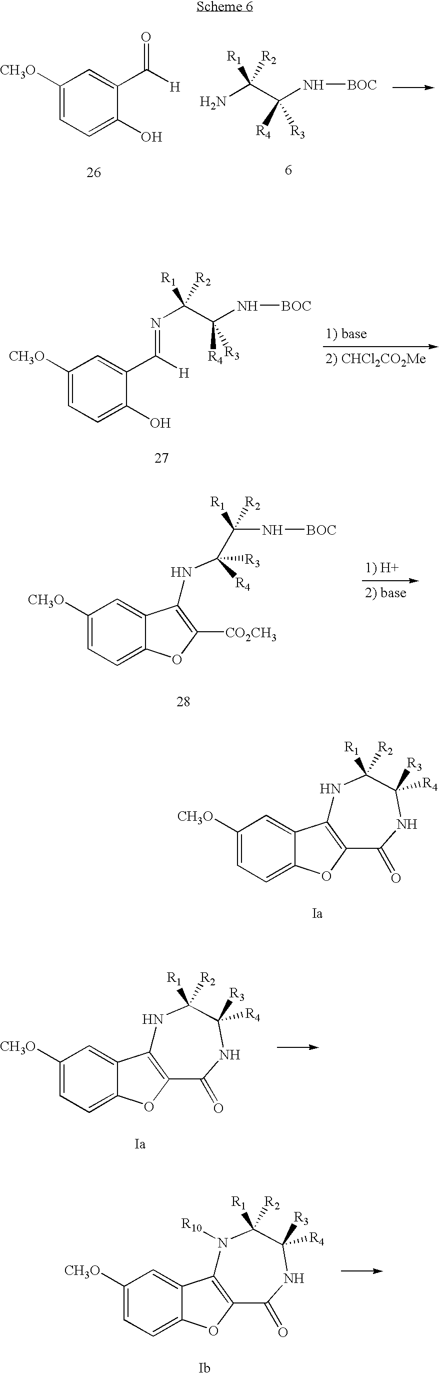

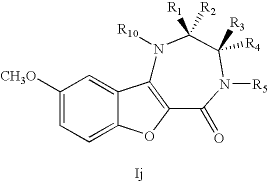

Further derivatization of these compounds can be conducted in a manner described in Scheme 1 to functionalize the nitrogen with R₁₀, to provide structures analogous to 1b. They can also be reacted as in Scheme 4 to derivatize R₈=methoxy and provide structures analogous to 1c. Further derivatization as outlined in Scheme 7 is also possible to introduce an R₅ group resulting in structures analogous to 1j.

### Derivatization of Diazepine Ring

The synthesis of compounds of structure 6 is given is Scheme 7.

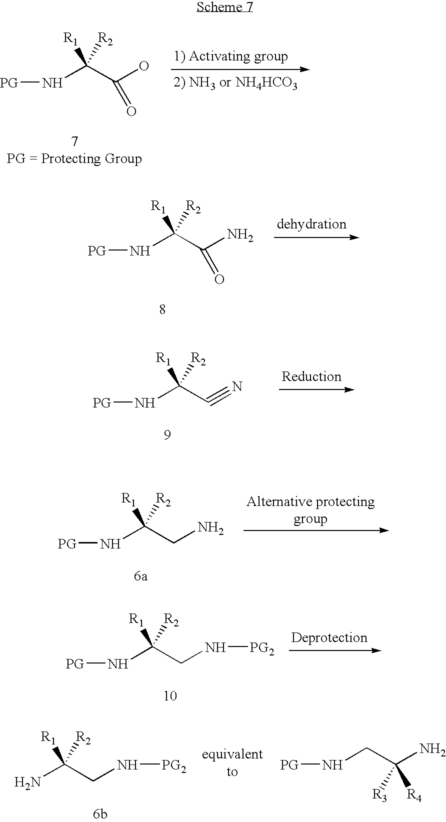

Compounds 6a and 6b are derived from the commercially available amino acids 7, either L or D, to impart stereochemistry. These compounds are commercially available with suitable protecting groups such as BOC or CBZ. The compound 7 is reacted with an activating group to afford a mixed anhydride, acid chloride, or other activated functionality, followed by an ammonia source such as gaseous ammonia, ammonium hydroxide, or ammonium bicarbonate. This provides protected amino amides of formula 8. The activating group may be an alkylchloroformate or anhydride, and the reaction is in suitable inert solvent such as dioxane, TBF or the like, in the presence of a non-nucleophilic base such as triethylamine if needed, at a temperature of −20° C. to 25° C. The intermediate activated ester is not isolated, but reacted directly with the ammonia source. This amide 8 is dehydrated using a reagent such as cyanuric chloride (Tet. Lett. 1997;38(24):4221) to afford the cyano derivative 9, in a suitable solvent such as dimethylformamide (DMF), followed by aqueous workup. This compound is then reduced using borane, lithium aluminum hydride or the like, in ether or THF, at a temperature of −10° C. to 80° C., under an inert atmosphere. The resulting amine 6a may be used directly as in Scheme 1, or may be further reacted to afford an amine of structure 6b. This is done by protecting the newly generated amine with a protecting group different than that of the original amine to afford the di-protected-di-amine 9. For example, if CBZ is the original protecting group of compound 7, the newly introduced protecting group could be BOC. The reaction would involve reacting 6a with BOC anhydride in dioxane in the presence of base as necessary, at a temperature of −10° C. to 25° C. The original protecting group of 9 is then removed under appropriate conditions such as catalytic hydrogenation for removal of the CBZ group. This provides compounds of structure 6b which are then used as in Scheme 1.

The compounds 6a or 6b described can be used in the synthesis of the substituted diazepine ring of structure as shown in Scheme 1 and Scheme 3. Alternatively, the commercially available ethylenediamine can be used for unsubstituted ring formation. The mono-protection of ethylenediamine with the BOC group is described in Syn. Comm. 1990;20(16):2559.

Within the compounds described the amide functionality can be derivatized as illustrated in Scheme 8.

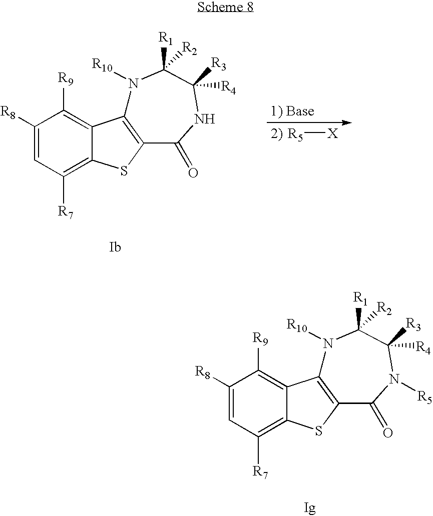

The compound 1b can be treated with a suitable base such as sodium hydride, KHMDS, or the like, followed by an alkylating agent such as methyl iodide to give derivative 1g. It can also be acylated with an acid chloride, anhydride, or other activated carbonyl.

Compounds of the generic structure 1 can also be acylated on nitrogen as illustrated in Scheme 9.

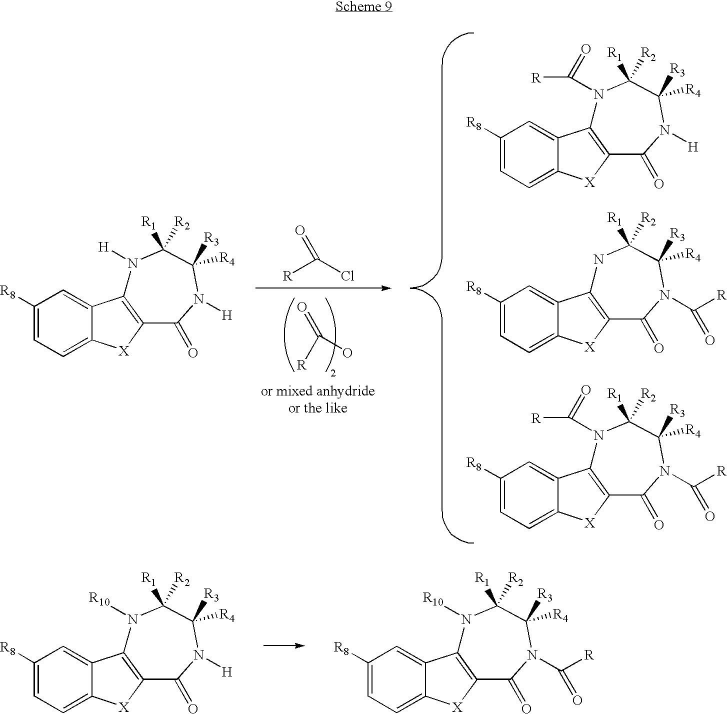

Reaction conditions can be varied to allow formation of one to three of the products. When R₁₀ is not H, acylation can be directed to the amide nitrogen.

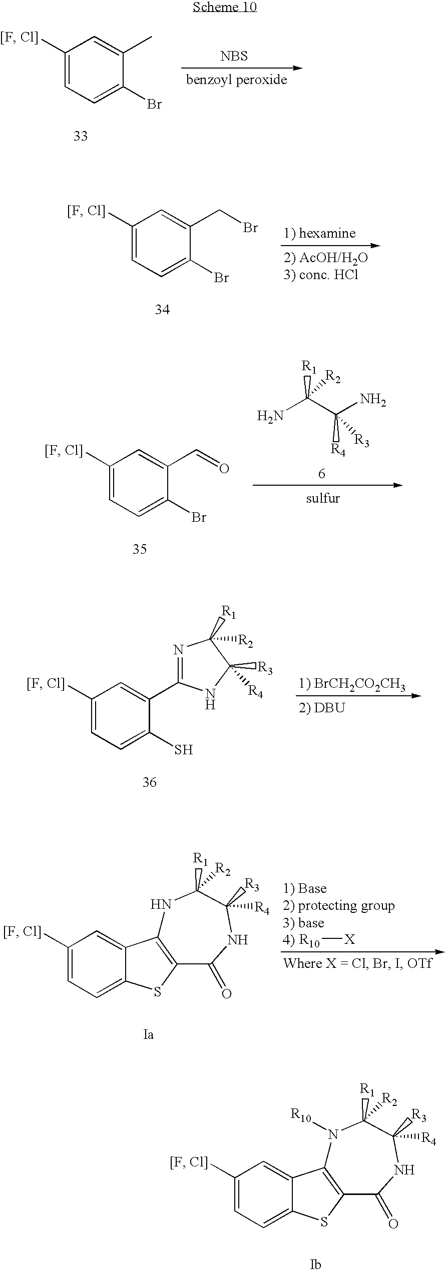

An alternative route when the EWG in Scheme 5 is fluorine is possible. This scheme can also be used in the EWG is chlorine. This is shown in Scheme 10. Thus, the commercially available compound 33 is brominated and produce compound 34, which is then treated with hexamine and the resulting ammonium compound hydrolyzed to afford aldehyde 35. Reactions analogous to those described in Scheme 3 and Scheme 1 are employed to transform 35 to 36 and so on into 1a and 1b.

### Alternative Two-Ring Core Structures

An alternative core ring structure useful in these derivatives is the isoquinoline which is synthesized as shown in Scheme 11.

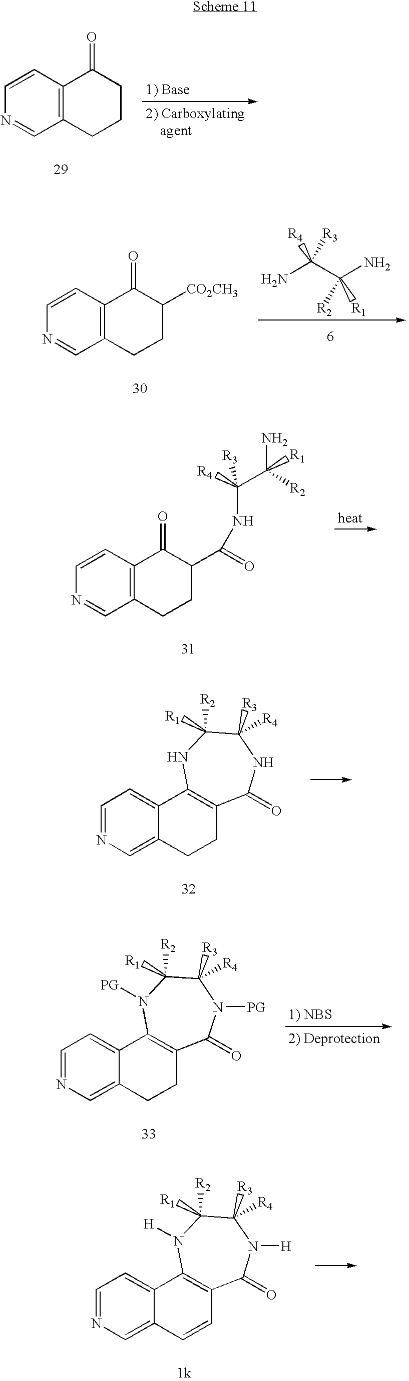

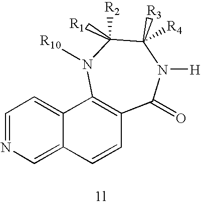

Thus the starting compound 29 (Syn. Comm. 1996;26:2305) is treated with a base such as lithium hexamethylenedisilazide, LDA, or the like, and a carboxylating reagent such as methyl cyanoformate at low temperature to provide 30. The amide 31 is made by reaction with diamine 6 by heating a mixture of the two compounds neat, which is then cyclized to 32 by refluxing in acetonitrile or another appropriate solvent. Both of the seven-membered ring nitrogens are protected with a suitable protecting group such as BOC to give 33, which increases compound solubility in a solvent such as carbon tetrachloride necessary to effect the subsequent radical aromatization using NBS and benzoyl peroxide. The protecting groups are removed under appropriate conditions (eg, treatment with an acid such as TFA or HCl for a BOC group) to give the product 1k. The compound can be subjected to reaction conditions such as described in Scheme 1 to further derivatize and introduce an R₁₀ substituent as in 11.

Conditions within the description of Schemes 1-11 and variations in the descriptions are known or can readily be determined from analogous reactions known to one skilled in the art.

The following abbreviations are used: Me=methyl; Bz=benzyl; Pr=propyl; BOC=CBZ=EtOAc=ethyl acetate; THF=tetrahydrofuran; MeOH=methanol.

Reactions in Schemes 1-11 are exemplified by the following general methods.

General Method 1-1

An equimolar amount of sulfoxide ester 1 and mono-protected diamine 6 are stirred together in distilled THF at room temperature to reflux for a period of 15 minutes to 18 hours. If ethylene diamine is the diamine used, mono-protection may not be necessary. The reaction is cooled if necessary, and the solid filtered. In some cases this solid is the product (as determined by mass spectral analysis TLC, and NMR), and can be used in the next step. If it is a mixture the entire reaction (solid and filtrate) is combined and chromatographed (SiO2, eluting 1:1 EtOAC/CH2Cl2+1-5% MeOH or the like) to isolate the product. Staining with iodine may be necessary to visualize the TLC plate.

The following compounds were made by this general method:

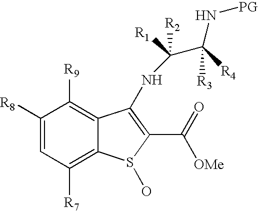

| Ex. | R₁ | R₂ | R₃ | R₄ | PG | R₇ | R₈ | R₉ | Mass Spec |
|---|---|---|---|---|---|---|---|---|---|
| 2a | H | H | H | H | BOC | H | OMe | H | m + 1 = 397 |
| 2b | H | H | H | Me | BOC | H | OMe | H | m + 1 = 411 |
| 2c | H | H | Me | H | BOC | H | OMe | H | m + 1 = 411 |
| 2d | H | H | H | Bz | BOC | H | OMe | H | m + 1 = 487 |
| 2e | H | H | Bz | H | BOC | H | OMe | H | m + 1 = 487 |
| 2f | H | H | H | i-Pr | BOC | H | OMe | H | m + 1 = 439 |
| 2g | H | H | i-Pr | H | BOC | H | OMe | H | m + 1 = 439 |
| 2h | Me | H | H | H | BOC | H | OMe | H | m + 1 = 411 |
| 2I | H | Me | H | H | BOC | H | OMe | H | m + 1 = 411 |
| 2j | Bz | H | H | H | BOC | H | OMe | H | m + 1 = 487 |
| 2k | H | Bz | H | H | BOC | H | OMe | H | m + 1 = 487 |
| 2l | i-Pr | H | H | H | BOC | H | OMe | H | m + 1 = 439 |
| 2m | H | i-Pr | H | H | BOC | H | OMe | H | m + 1 = 439 |
| 2n | H | H | H | H | H | H | OMe | H | m + 1 = 297 |

Names of compounds:

3-(2-tert-Butoxycarbonylamino-ethylamino)-5-methoxy-1-oxo-1H-1λ⁴-benzo[b]thiophene-2-carboxylic acid methyl ester (2a);

(S)-3-(2-tert-Butoxycarbonylamino-propylamino)-5-methoxy-1-oxo-1H-1λ⁴-benzo[b]thiophene-2-carboxylic acid methyl ester (2b);

(R)-3-(2-tert-Butoxycarbonylamino-propylamino)-5-methoxy-1-oxo-1H-1λ⁴-benzo[b]thiophene-2-carboxylic acid methyl ester (2c);

(S)-3-(2-tert-Butoxycarbonylamino-3-phenyl-propylamino)-5′-methoxy-1-oxo-1H-1λ⁴-benzo[b]thiophene-2-carboxylic acid methyl ester (2d);

(R)-3-(2-tert-Butoxycarbonylamino-3-phenyl-propylamino)-5-methoxy-1-oxo-1H-1λ⁴-benzo[b]thiophene-2-carboxylic acid methyl ester (2e);

(S)-3-(2-tert-Butoxycarbonylamino-3-methyl-butylamino)-5-methoxy-1-oxo-1H-1λ⁴-benzo[b]thiophene-2-carboxylic acid methyl ester (2f);

(R)-3-(2-tert-Butoxycarbonylamino-3-methyl-butylamino)-5-methoxy-1-oxo-1H-1λ⁴-benzo[b]thiophene-2-carboxylic acid methyl ester (2g);

(S)-3-(2-tert-Butoxycarbonylamino-1-methyl-ethylamino)-5-methoxy-1-oxo-1H-1λ⁴-benzo[b]thiophene-2-carboxylic acid methyl ester (2h);

(R)-3-(2-tert-Butoxycarbonylamino-1-methyl-ethylamino)-5-methoxy-1-oxo-1H-1λ⁴-benzo[b]thiophene-2-carboxylic acid methyl ester (2i);

(S)-3-(1-Benzyl-2-tert-butoxycarbonylamino-ethylamino)-5-methoxy-1-oxo-1H-1λ⁴-benzo[b]thiophene-2-carboxylic acid methyl ester (2j);

(R)-3-(1-Benzyl-2-tert-butoxycarbonylamino-ethylamino)-5-methoxy-1-oxo-1H-1λ⁴-benzo[b]thiophene-2-carboxylic acid methyl ester (2k);

(S)-3-[1-(tert-Butoxycarbonylamino-methyl)-2-methyl-propylamino]-5-methoxy-1-oxo-1H-1λ⁴-benzo[b]thiophene-2-carboxylic acid methyl ester (2l);

(R)-3-[1-(tert-Butoxycarbonylamino-methyl)-2-methyl-propylamino]-5-methoxy-1-oxo-1H-1λ⁴-benzo[b]thiophene-2-carboxylic acid methyl ester (2m); and

3-(2-Amino-ethylamino)-5-methoxy-1-oxo-H-1λ⁴-benzo[b]thiophene-2-carboxylic acid methyl ester (2n).

General Method 1-2

The sulfoxide group on compounds 2 is removed reductively, either by hydrogenation or by treatment with TMSCl/NaI (J. Med. Chem. 1981:683) to afford the benzothiophene core.

Sulfoxide compound 2 is dissolved in a small amount of acetonitrile, and three equivalents of sodium iodide is added. The reaction is placed under a nitrogen atmosphere and cooled in an ice bath. Trimethylsilylchloride is added to the reaction dropwise which produces a brown color, and the reaction is allowed to come to ambient temperature over 4 to 24 hours. It is then diluted with diethyl ether, washed with 10% aqueous sodium thiosulfate (×2), the water layers back-extracted with ether, and the combined organic layers washed with brine, dried (MgSO₄ or Na₂SO₄), and concentrated. The crude material can be carried on to the next reaction.

The following compounds were made by this general method:

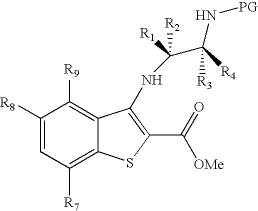

| Ex. | R₁ | R₂ | R₃ | R₄ | PG | R₇ | R₈ | R₉ | Mass Spec |
|---|---|---|---|---|---|---|---|---|---|
| 3a | H | H | H | H | BOC | H | OMe | H | m + 1 = 381 |
| 3b | H | H | H | Me | BOC | H | OMe | H | m + 1 = 395 |
| 3c | H | H | Me | H | BOC | H | OMe | H | m + 1 = 395 |
| 3d | H | H | H | Bz | BOC | H | OMe | H | m + 1 = 471 |
| 3e | H | H | Bz | H | BOC | H | OMe | H | m + 1 = 471 |
| 3f | H | H | H | iPr | BOC | H | OMe | H | m + 1 = 423 |
| 3g | H | H | i-Pr | H | BOC | H | OMe | H | m + 1 = 423 |
| 3h | Me | H | H | H | BOC | H | OMe | H | m + 1 = 411 |
| 3I | H | Me | H | H | BOC | H | OMe | H | m + 1 = 411 |
| 3j | Bz | H | H | H | BOC | H | OMe | H | m + 1 = 471 |
| 3k | H | Bz | H | H | BOC | H | OMe | H | m + 1 = 471 |
| 3l | i-Pr | H | H | H | BOC | H | OMe | H | m + 1 = 423 |
| 3m | H | IPr | H | H | BOC | H | OMe | H | m + 1 = 423 |
| 3n | H | H | H | H | H | H | OMe | H | m + 1 = 281 |

Names of compounds:

3-(2-tert-Butoxycarbonylamino-ethylamino)-5-methoxy-benzo[b]thiophene-2-carboxylic acid methyl ester (3a);

(S)-3{2-tert-Butoxycarbonylamino-propylamino)-5-methoxy-benzo[b]thiophene-2 carboxylic acid methyl ester (3b);

(R)-3-(2-tert-Butoxycarbonylamino-propylamino)-5-methoxy-benzo[b]thiophene-2-carboxylic acid methyl ester (3c);

(S)-3-(2-tert-Butoxycarbonylamino-3-phenyl-propylamino)-5-methoxy-benzo[b]thiophene-2-carboxylic acid methyl ester (3d);

(R)-3-(2-tert-Butoxycarbonylamino-3-phenyl-propylamino)-5-methoxy-benzo[b]thiophene-2-carboxylic acid methyl ester (3e);

(S)-3-(2-tert-Butoxycarbonylamino-3-methyl-butylamino)-5-methoxy-benzo[b]thiophene-2-carboxylic acid methyl ester (3f);

(R)-3-(2-tert-Butoxycarbonylamino-3-methyl-butylamino)-5-methoxy-benzo[b]thiophene-2-carboxylic acid methyl ester (3g);

(S)-3-(2-tert-Butoxycarbonylamino-1-methyl-ethylamino)-5-methoxy-benzo[b]thiophene-2-carboxylic acid methyl ester (3h);

(R)-3-(2-tert-Butoxycarbonylamino-1-methyl-ethylamino)-5-methoxy-benzo[b]thiophene-2-carboxylic acid methyl ester (3i);

(S)-3-(1-Benzyl-2-tert-butoxycarbonylamino-ethylamino)-5-methoxy-benzo[b]thiophene-2-carboxylic acid methyl ester (3j);

(R)-3-[1-Benzyl-2-tert-butoxycarbonylamino-ethylamino)-5-methoxy-benzo[b]thiophene-2-carboxylic acid methyl ester (3k);

(S)-3-[1-(tert-Butoxycarbonylamino-methyl)-2-methyl-propylamino]-5-methoxy-benzo[b]thiophene-2-carboxylic acid methyl ester (3l);

(R)-3-[1-(tert-Butoxycarbonylamino-methyl)-2-methyl-propylamino]-5-methoxy-benzo[b]thiophene-2-carboxylic acid methyl ester (3m); and

3-(2-Amino-ethylamino)-5-methoxy-benzo[b]thiophene-2-carboxylic acid methyl ester (3n).

General Method 1-3-1

The BOC-protected amine compound 3 was dissolved in dichloromethane or diethyl ether, chilled in an ice bath under a nitrogen atmosphere, and to this was added an excess of trifluoroacetic acid (TFA). The reaction was allowed to come to ambient temperature over 4 to 24 hours, then concentrated. The residue was dissolved in toluene, re-concentrated, and this procedure repeated ×2. Finally the reaction was suspended in a small amount of diethyl ether, stirred, and the solid filtered. This solid was the product

General Method 1-3-2

The BOC-protected amine compound 3 was treated as in General Method 1-3-1 with 1.0-4.0 M HCl in diethyl ether used as an acid in place of TFA.

The following compounds were made by these methods:

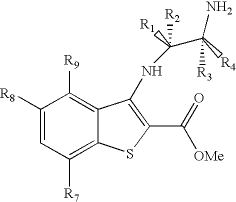

| Example | | | | | | | | | |
|---|---|---|---|---|---|---|---|---|---|
| No. | R₁ | R₂ | R₃ | R₄ | R₇ | R₈ | R₉ | Method | Mass Spec |
| 4a | H | H | H | H | H | OMe | H | 1-3-2 | m + 1 = 281 |
| 4b | H | H | H | Me | H | OMe | H | 1-3-2 | m + 1 = 295 |
| 4c | H | H | Me | H | H | OMe | H | 1-3-2 | m + 1 = 295 |
| 4d | H | H | H | Bz | H | OMe | H | 1-3-2 | m + 1 = 371 |
| 4e | H | H | Bz | H | H | OMe | H | 1-3-2 | m + 1 = 371 |
| 4f | H | H | H | i-Pr | H | OMe | H | 1-3-1 | m + 1 = 323 |
| 4g | H | H | i-Pr | H | H | OMe | H | 1-3-1 | m + 1 = 323 |
| 4h | Me | H | H | H | H | OMe | H | 1-3-2 | m + 1 = 295 |
| 4i | H | Me | H | H | H | OMe | H | 1-3-2 | m + 1 = 295 |
| 4j | Bz | H | H | H | H | OMe | H | 1-3-2 | m + 1 = 371 |
| 4k | H | Bz | H | H | H | OMe | H | 1-3-2 | m + 1 = 371 |
| 4l | i-Pr | H | H | H | H | OMe | H | 1-3-1 | m + 1 = 323 |
| 4m | H | i-Pr | H | H | H | OMe | H | 1-3-1 | m + 1 = 323 |

Names of compounds:

3-(2-Amino-ethylamino)-5-methoxy-benzo[b]thiophene-2-carboxylic acid methyl ester (4a);

(S)-3-(2-Amino-propylamino)-5-methoxy-benzo[b]thiophene-2-carboxylic acid methyl ester (4b);

(R)-3-(2-Amino-propylamino)-5-methoxy-benzo[b]thiophene-2-carboxylic acid methyl ester (4c);

(S)-3-(2-Amino-3-phenyl-propylamino)-5-methoxy-benzo[b]thiophene-2-carboxylic acid methyl ester (4d);

(R)-3-(2-Amino-3-phenyl-propylamino)-5-methoxy-benzo[b]thiophene-2-carboxylic acid methyl ester (4e);

(S)-3-(2-Amino-3-methyl-butylamino)-5-methoxy-benzo[b]thiophene-2-carboxylic acid methyl ester (4f);

(R)-3-(2-Amino-3-methyl-butylamino)-5-methoxy-benzo[b]thiophene-2-carboxylic acid methyl ester (4g);

(S)-3-(2-Amino-1-methyl-ethylamino)-5-methoxy-benzo[b]thiophene-2-carboxylic acid methyl ester (4h);

(R)-3-(2-Amino-1-methyl-ethylamino)-5-methoxy-benzo[b]thiophene-2-carboxylic acid methyl ester (4i);

(S)-3-(2-Amino-1-benzyl-ethylamino)-5-methoxy-benzo[b]thiophene-2-carboxylic acid methyl ester (4j);

(R)-3-(2-Amino-1-benzyl-ethylamino)-5-methoxy-benzo[b]thiophene-2-carboxylic acid methyl ester (4k);

(S)-3-(1-Aminomethyl-2-methyl-propylamino)-5-methoxy-benzo[b]thiophene-2-carboxylic acid methyl ester (4l); and

(R)-3-(1-Aminomethyl-2-methyl-propylamino)-5-methoxy-benzo[b]thiophene-2-carboxylic acid methyl ester (4m).

General Method 1-4-1

The amino esters 4 can be cyclized into a seven-membered ring by heating, most often in the presence of base, to afford the lactam compound 1a. Heating in methanol may be sufficient to effect cyclization is some cases.

The amino ester 4 is dissolved in methanol under a nitrogen atmosphere, then treated with 3 to 10 equivalents of sodium hydride. The reaction is refluxed for 1 to 24 hours, then cooled, concentrated, and the residue taken up in EtOAc. This solution is washed with water, brine, and dried (MgSO₄ or Na₂SO₄), and the solvent removed under reduced pressure. Alternatively the reaction can be neutralized with an acid such as 1.0N HCl, concentrated, partitioned between water and an organic solvent such as EtOAc, the organic layer washed with brine, dried and concentrated. The residue can often be tritrated with diethyl ether to afford the product. Alternatively, if necessary, the reaction can be purified by column chromatography (SiO₂ eluting with CH₂Cl₂/MeOH 99:1 to 9:1; 1:1 EtOAc/CH₂Cl₂+1%-5% MeOH or the like).

General Method 12

A flask containing methanol is treated with 3 to 10 equivalents sodium pellets (washed in hexanes or toluene), and stirred until dissolved, under nitrogen. To this solution is added a methanolic solution of the amino ester 4, and the reaction refluxed 1 to 24 hours, cooled, quenched with water, then concentrated. The residue is partitioned between water and EtOAc, the organic layer separated and washed with brine, dried, and concentrated. Alternatively the reaction can be neutralized with an acid such a 1.0N HCl, concentrated, partitioned between water and an organic solvent such as EtOAc, the organic layer washed with brine, dried and concentrated. The product is purified as in Method 1-4-1. It is also possible to isolate the product from the reaction mixture if the pH is adjusted to acidic (˜pH3) and the reaction mixture cooled to form crystals.

The following compounds were made by these methods:

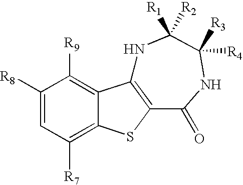

| Example | R₁ | R₂ | R₃ | R₄ | R₇ | R₈ | R₉ | Method | Mass Spec |
|---|---|---|---|---|---|---|---|---|---|
| B | H | H | H | H | H | OMe | H | 1-4-2 | m + 1 = 249 |
| HH2 | H | H | H | Me | H | OMe | H | 1-4-2 | m + 1 = 263 |
| LL2 | H | H | Me | H | H | OMe | H | 1-4-2 | m + 1 = 263 |
| DD3 | H | H | H | Bz | H | OMe | H | 1-4-2 | m + 1 = 339 |
| KK2 | H | H | Bz | H | H | OMe | H | 1-4-2 | m + 1 = 339 |
| JJ2 | H | H | H | i-Pr | H | OMe | H | 1-4-1 | m + 1 = 291 |
| II2 | H | H | i-Pr | H | H | OMe | H | 1-4-1 | m + 1 = 291 |
| MM2 | Me | H | H | H | H | OMe | H | 1-4-2 | m + 1 = 263 |
| NN2 | H | Me | H | H | H | OMe | H | 1-4-2 | m + 1 = 263 |
| PP2 | Bz | H | H | H | H | OMe | H | 1-4-2 | m + 1 = 339 |
| OO2 | H | Bz | H | H | H | OMe | H | 1-4-2 | m + 1 = 339 |
| QQ2 | i-Pr | H | H | H | H | OMe | H | 1-4-1 | m + 1 = 291 |
| OO | H | H | Me | Me | H | OMe | H | 1-4-2 | m + 1 = 277 |

Names of compounds:

3-Methoxy-5,6,7,8-tetrahydro-10-thia-5,8-diaza-benzo[a]azulen-9-one (B);

3-Methoxy-7-methyl-5,6,7,8-tetrahydro-10-thia-5,8-diaza-benzo[a]azulen-9-one (S) (HH2);

3-Methoxy-7-methyl-5,6,7,8-tetrahydro-10-thia-5,8-diaza-benzo[a]azulen-9-one (R) (LL2);

7-Benzyl-3-methoxy-5,6,7,8-tetrahydro-10-thia-5,8-diaza-benzo[a]azulen-9-one (S) (DD3);

7-Benzyl-3-methoxy-5,6,7,8-tetrahydro-10-thia-5,8-diaza-benzo[a]azulen-9-one (R) (KK2);

7-Isopropyl-3-methoxy-5,6,7,8-tetrahydro-10-thia-5,8-diaza-benzo[a]azulen-9-one (S) (JJ2);

7-Isopropyl-3-methoxy-5,6,7,8-tetrahydro-10-thia-5,8-diaza-benzo[a]azulen-9-one (R) (II2);

3-Methoxy-6-methyl-5,6,7,8-tetrahydro-10-thia-5,8-diaza-benzo[a]azulen-9-one (S) (MM2);

3-Methoxy-6-methyl-5,6,7,8-tetrahydro-10-thia-5,8-diaza-benzo[a]azulen-9-one (R) (NN2);

6-Benzyl-3-methoxy-5,6,7,8-tetrahydro-10-thia-5,8-diaza-benzo[a]azulen-9-one (S) (PP2);

6-Benzyl-3-methoxy-5,6,7,8-tetrahydro-10-thia-5,8-diaza-benzo[a]azulen-9-one (R) (OO2);

3-Methoxy-6-iso-propyl-5,6,7,8-tetrahydro-10-thia-5,8-diaza-benzo[a]azulen-9-one (S) (QQ2); and

3-Methoxy-7,7-dimethyl-5,6,7,8-tetrahydro-10-thia-5,8-diaza-benzo[a]azulen-9-one (OO).

Scheme 1 Step 5, Scheme 2 Step 4. This reaction also applies to compounds from Schemes 3, 4, 5, 6, 9, 10, and 11.

The selective alkylation is conducted as follows:

General Method 1-5-1

Under an argon atmosphere a solution of 1a in TBF is cooled to 0° C. To this solution is added n-butyl lithium (2 equivalents) and the mixture stirred for 15 minutes. t-Butyl dimethyl chlorosilane is added, then the mixture stirred at room temperature for 15 minutes. Alkyl halide (5 equivalents) is added and the mixture stirred for 3 hours. A saturated aqueous sodium bicarbonate solution is added to quench, and the aqueous layer extracted the EtOAc. The combined organic layers are washed with brine, dried over magnesium sulfate, and concentrated under reduced pressure. The residue is purified by flash column chromatography to give the product.

General Method 1-5-2

Under an argon atmosphere a solution of 1a in DMF is cooled to 0° C. To the solution is added t-butyldimethylchlorosilane (1.1 equivalents) and the mixture stirred for 5 minutes. Sodium hydride (60% dispersion in mineral oil, 2.2 equivalents) is added, and the mixture stirred at room temperature for 10 minutes. Alkyl halide (1.5 equivalents) is added and the mixture stirred for 2 hours. A saturated aqueous sodium bicarbonate solution was used to quench the reaction, and the reaction extracted with EtOAc. The combined organic layers are washed with water and brine, dried over magnesium sulfate, and concentrated under reduced pressure. The residue is purified by flash column chromatography (60-100% EtOAc/CHCl₃ then 0-10% THF/EtOAc) and recrystallized in EtOAc to give the product.

The following compounds were made:

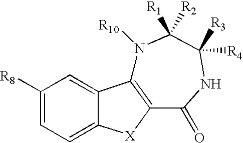

| | | | | | | | | | Mass Spectra |
|---|---|---|---|---|---|---|---|---|---|
| Example | R₁ | R₂ | R₃ | R₄ | R₈ | R₁₀ | X | General | (m + 1) |
| II | H | H | H | H | OMe | n-propyl | S | 1-5-2 | 291 |
| JJ | H | H | H | H | OMe | n-butyl | S | 1-5-1 | 305 |
| KK | H | H | H | H | OMe | i-butyl | S | 1-5-1 | 305 |
| LL | H | H | H | H | OMe | 2-methyl-allyl | S | 1-5-1 | 317 |
| MM | H | H | H | H | OMe | i-propyl | S | 1-5-2 | 319 |
| FF3 | H | H | H | H | OMe | Methyl | S | 1-5-1 | 263 |
| HH3 | H | H | H | H | OMe | Allyl | S | 1-5-1 | 289 |
| II3 | H | H | H | H | OMe | Ethyl-aceto | S | 1-5-1 | 335 |
| JJ3 | H | H | H | H | OMe | Acetic-acid | S | 1-5-1 | 307 |
| KK3 | H | H | H | H | OMe | Benzyl | S | 1-5-1 | 339 |
| LL3 | H | H | H | H | OMe | 4-chloro-benzyl | S | 1-5-1 | 373 |
| MM3 | H | H | H | H | OMe | 2-chloro-allyl | S | 1-5-1 | 323 |
| OO3 | H | H | H | H | OMe | 4-methyl-benzyl | S | 1-5-1 | 353 |
| C | H | H | H | H | OMe | 3-methoxy- | S | 1-5-1 | 369 |
| | | | | | | benzyl | | | |
| QQ3 | H | H | H | H | OMe | 4-methoxy- | S | 1-5-1 | 369 |
| | | | | | | benzyl | | | |
| RR3 | H | H | H | H | OMe | 2-chloro-benzyl | S | 1-5-1 | 373 |
| SS3 | H | H | H | H | OMe | 4-nitro-benzyl | S | 1-5-1 | 384 |
| TT3 | H | H | H | H | OMe | 3-methyl-benzyl | S | 1-5-1 | 353 |
| UU3 | H | H | H | H | OMe | 3-chloro-benzyl | S | 1-5-1 | 373 |
| VV3 | H | H | H | H | OMe | 3-nitro-benzyl | S | 1-5-1 | 384 |
| WW3 | H | H | H | H | OMe | 2-methyl-benzyl | S | 1-5-1 | 353 |
| XX3 | H | H | H | H | OMe | 3-methyl-butane | S | 1-5-2 | 319 |
| YY3 | H | H | H | H | OMe | 2-methoxy- | S | 1-5-1 | 369 |
| | | | | | | benzyl | | | |
| ZZ3 | H | H | H | H | OMe | 4-tetra-azole- | S | 1-5-1 | 407 |
| | | | | | | benzyl | | | |
| AA4 | H | H | H | H | OMe | 4-nitrile-benzyl | S | 1-5-1 | 378 |
| BB4 | H | H | H | H | OMe | Ethyl | S | 1-5-2 | 277 |
| DD4 | H | H | H | H | OMe | (3-pyridyl)- | S | 1-5-1 | 340 |
| | | | | | | methyl | | | |
| O | H | H | H | methyl | OMe | m-OMe-benzyl | S | 1-5-2 | 383 |
| R | H | H | benzyl | H | OMe | n-Propyl | S | 1-5-2 | 381 |
| WW | H | H | H | methyl | OMe | n-propyl | S | 1-5-2 | 305 |
| XX | H | H | H | methyl | OMe | 2-methyl-allyl | S | 1-5-1 | 317 |
| YY | H | H | H | benzyl | OMe | n-propyl | S | 1-5-2 | 381 |
| ZZ | H | H | H | benzyl | OMe | 2-methyl-allyl | S | 1-5-2 | 393 |
| BB2 | H | H | benzyl | H | OMe | 2-methyl-allyl | S | 1-5-2 | 393 |
| CC2 | H | H | benzyl | H | OMe | m-OMe-benzyl | S | 1-5-2 | 459 |
| DD2 | H | H | H | benzyl | OMe | m-OMe-benzyl | S | 1-5-2 | 459 |
| EE2 | H | H | methyl | H | OMe | m-OMe-benzyl | S | 1-5-2 | 383 |
| FF2 | H | H | H | propyl | OMe | n-propyl | S | 1-5-2 | 333 |
| GG2 | H | H | H | propyl | OMe | m-OMe-benzyl | S | 1-5-1 | 411 |
| K | H | H | methyl | methyl | OMe | Methoxy-benzyl | S | 1-5-1 | 397 |
| TT | H | H | methyl | methyl | OMe | n-propyl | S | 1-5-2 | 319 |
| FF | H | H | methyl | methyl | H | i-butyl | S | 1-5-2 | 303 |
| PP | H | H | H | H | H | 2-methyl-allyl | S | 1-5-2 | 273 |
| QQ | H | H | methyl | methyl | H | Methoxy-benzyl | 5 | 1-5-1 | 367 |
| RR | H | H | methyl | methyl | H | 2-methyl-allyl | S | 1-5-2 | 301 |
| SS | H | H | methyl | methyl | H | i-butyl | S | 1-5-2 | 303 |
| UU | H | H | methyl | methyl | OMe | 2-methyl-allyl | S | 1-5-2 | 331 |
| Q | H | H | H | H | NO₂ | 2-methyl-allyl | S | 1-5-1 | 318 |
| S | H | H | H | H | NO₂ | n-Propyl | S | 1-5-2 | 306 |
| T | H | H | H | H | NO₂ | i-Butyl | S | 1-5-1 | 320 |
| U | H | H | H | H | NO₂ | Allyl | S | 1-5-1 | 304 |
| V | H | H | H | H | NO₂ | 3-methoxy- | S | 1-5-1 | 384 |
| | | | | | | benzyl | | | |
| W | H | H | H | H | NO₂ | 3-nitro-benzyl | S | 1-5-1 | 399 |
| X | H | H | H | H | NO₂ | Benzyl | S | 1-5-1 | 354 |
| Y | H | H | H | H | NO₂ | 3-chloro-benzyl | S | 1-5-1 | 388 |
| BB | H | H | H | H | NO₂ | Ethyl | S | 1-5-2 | 292 |
| CC | H | H | H | H | NO₂ | 3-methyl- | S | 1-5-2 | 332 |
| | | | | | | 2-butene | | | |
| EE | H | H | H | H | NO₂ | 2-methyl-allyl | O | 1-5-2 | 302 |
| GG | H | H | H | H | NO₂ | 2-fluoro-benzyl | S | 1-5-1 | 372 |
| DD | H | H | H | H | NO₂ | n-butyl | S | 1-5-2 | 320 |
| H | H | H | H | H | OMe | Ethyl | O | 1-5-2 | 261 |
| I | H | H | H | H | OMe | 2-Methyl-allyl | O | 1-5-2 | 287 |
| N | H | H | H | H | OMe | Iso-butyl | O | 1-5-2 | 289 |
| XX4 | H | H | H | H | Cl | n-propyl | O | 1-5-2 | 279 |
| YY4 | H | H | H | H | Cl | n-butyl | O | 1-5-2 | 293 |
| ZZ4 | H | H | H | H | Cl | Iso-pentyl | O | 1-5-2 | 307 |
| BB5 | H | H | H | H | Br | 2-methyl-allyl | O | 1-5-2 | 336 |
| CC5 | H | H | H | H | F | 2-methyl-allyl | S | 1-5-2 | 291 |
| DD5 | H | H | H | H | F | n-butyl | S | 1-5-2 | 293 |
| EE5 | H | H | H | H | F | Allyl | S | 1-5-2 | 277 |
| FF5 | H | H | H | H | F | Benzyl | S | 1-5-2 | 327 |
| GG5 | H | H | H | H | Br | Allyl | S | 1-5-2 | 322 |
| HH5 | H | H | H | H | Br | n-butyl | O | 1-5-2 | 338 |
| II5 | H | H | H | H | Br | Benzyl | O | 1-5-2 | 372 |
| KK5 | H | H | H | H | Cl | 2-methyl-allyl | S | 1-5-2 | 307 |
| LL5 | H | H | H | H | Cl | Benzyl | S | 1-5-2 | 343 |
| MM5 | H | H | H | H | Cl | n-butyl | S | 1-5-2 | 309 |
| NN5 | H | H | H | H | Cl | Allyl | S | 1-5-2 | 293 |
| OO5 | H | H | H | H | Br | 2-F-benzyl | O | 1-5-2 | 399 |
| PP5 | H | H | H | H | Br | 3-F-benzyl | O | 1-5-2 | 390 |
| QQ5 | H | H | H | H | Cl | 2-F-benzyl | S | 1-5-2 | 361 |
| RR5 | H | H | H | H | Cl | 3-Cl-benzyl | S | 1-5-2 | 378 |
| SS5 | H | H | H | H | Cl | 3-OMe-benzyl | S | 1-5-2 | 373 |
| TT5 | H | H | H | H | Cl | iso-pentenyl | S | 1-5-2 | 321 |
| VV5 | H | H | H | H | Cl | Benzyl | O | 1-5-2 | 327 |
| WW5 | H | H | H | H | F | iso-pentenyl | S | 1-5-2 | 305 |
| XX5 | H | H | H | H | F | 2-propyne | S | 1-5-2 | 275 |
| YY5 | H | H | H | H | OMe | i-pentyl | O | 1-5-2 | 303 |

Names of compounds:

3-Methoxy-5-propyl-5,6,7,8-tetrahydro-10-thia-5,8-diaza-benz[a]azulen-9-one (II);

5-Butyl-3-methoxy-5,6,7,8-tetrahydro-10-thia-5,8-diaza-benz[a]azulen-9-one (JJ);

5-Isobutyl-3-methoxy-5,6,7,8-tetrahydro-10-thia-5,8-diaza-benzo[a]azulen-9-one (KK);

3-Methoxy-5-(3-methyl-but-2-enyl)-5,6,7,8-tetrahydro-10-thia-5,8-diaza-benzo[a]azulen-9-one (LL);

3-Methoxy-5-(3-methyl-butyl)-5,6,7,8-tetrahydro-10-thia-5,8-diaza-benz[a]azulen-9-one (MM);

3-Methoxy-5-methyl-5,6,7,8-tetrahydro-10-thia-5,8-diaza-benzo[a]azulen-9-one (FF3);

5-Allyl-3-methoxy-5,6,7,8-tetrahydro-10-thia-5,8-diaza-benzo[a]azulen-9-one (HH3);

(3-Methoxy-9-oxo-6,7,8,9-tetrahydro-10-thia-5,8-diaza-benzora]azulen-5-yl)-acetic acid ethyl ester (II3);

(3-Methoxy-9-oxo-6,7,8,9-tetrahydro-10-thia-5,8-diaza-benzo[a]azulen-5-yl)-acetic acid (JJ3);

5-Benzyl-3-methoxy-5,6,7,8-tetrahydro-10-thia-5,8-diaza-benzo[a]azulen-9-one (KK3);

5-(4-Chloro-benzyl)-3-methoxy-5,6,7,8-tetrahydro-10-thia-5,8-diaza-benzo[a]azulen-9-one (LL3);

5-(2-Chloro-allyl)-3-methoxy-5,6,7,8-tetrahydro-10-thia-5,8-diaza-benzo[a]azulen-9-one (MM3);

3-Methoxy-5-(4-methyl-benzyl)-5,6,7,8-tetrahydro-10-thia-5,8-diaza-benzo[a]azulen-9-one (003);

3-Methoxy-5-(3-methoxy-benzyl)-5,6,7,8-tetrahydro-10-thia-5,8-diaza-benzo[a]azulen-9-one (C);

3-Methoxy-5-(4-methoxy-benzyl)-5,6,7,8-tetrahydro-10-thia-5,8-diaza-benzo[a]azulen-9-one (QQ3);

5-(2-Chloro-benzyl)-3-methoxy-5,6,7,8-tetrahydro-10-thia-5,8-diaza-benzo[a]azulen-9-one (RR3);

3-Methoxy-5-(4-nitro-benzyl)-5,6,7,8-tetrahydro-10-thia-5,8-diaza-benzo[a]azulen-9-one (SS3);

3-Methoxy-5-(3-methyl-benzyl)-5,6,7,8-tetrahydro-10-thia-5,8-diaza-benzo[a]azulen-9-one (TT3);

5-(3-Chloro-benzyl)-3-methoxy-5,6,7,8-tetrahydro-10-thia-5,8-diaza-benzo[a]azulen-9-one (UU3);

3-Methoxy-5-(3-nitro-benzyl)-5,6,7,8-tetrahydro-10-thia-5,8-diaza-benzo[a]azulen-9-one (VV3);

3-Methoxy-52-methyl-benzyl)-5,6,7,8-tetrahydro-10-thia-5,8-diaza-benzo[a]azulen-9-one (WW3);

5-(3-Methyl-butyl)-3-methoxy-5,6,7,8-tetrahydro-10-thia-5,8-diaza-benzo[a]azulen-9-one (XX3);

3-Methoxy-5-(2-methoxy-benzyl)-5,6,7,8-tetrahydro-10-thia-5,8-diaza-benzo[a]azulen-9-one (YY3);

3-Methoxy-5-[4-(2H-tetrazol-5-yl)-benzyl]-5,6,7,8-tetrahydro-10-thia-5,8-diaza-benz[a]azulen-9-one (ZZ3);

4-(3-Methoxy-8-methyl-9-oxo-6,7,8,9-tetrahydro-10-thia-5,8-diaza-benzo[a]azulen-5-ylmethyl)-benzonitrile (AA4);

5-Ethyl-3-methoxy-5,6,7,8-tetrahydro-10-thia-5,8-diaza-benzo[a]azulen-9-one (BB4);

3-Methoxy-5-pyridin-3-ylmethyl-5,6,7,8-tetrahydro-10-thia-5,8-diaza-benz[a]azulen-9-one (DD4);

3-Methoxy-5-(3-methoxy-benzyl)-7-methyl-5,6,7,8-tetrahydro-10-thia-5,8-diaza-benzo[a]azulen-9-one (O);

7-Benzyl-3-methoxy-5-propyl-5,6,7,8-tetrahydro-10-thia-5,8-diaza-benzo[a]azulen-9-one (R);

3-Methoxy-7-methyl-5-propyl-5,6,7,8-tetrahydro-10-thia-5,8-diaza-benzo[a]azulen-9-one (WW);

3-Methoxy-7-methyl-5-(2-methyl-allyl)-5,6,7,8-tetrahydro-10-thia-5,8-diaza-benzo[a]azulen-9-one (XX);

7-Benzyl-3-methoxy-5-propyl-5,6,7,8-tetrahydro-10-thia-5,8-diaza-benzo[a]azulen-9-one (YY);

7-Benzyl-3-methoxy-5-(2-methyl-allyl)-5,6,7,8-tetrahydro-10-thia-5,8-diaza-benzo[a]azulen-9-one (ZZ);

7-Benzyl-3-methoxy-5-(2-methyl-allyl)-5,6,7,8-tetrahydro-10-thia-5,8-diaza-benzo[a]azulen-9-one (BB2);

7-Benzyl-3-methoxy-5-(3-methoxy-benzyl)-5,6,7,8-tetrahydro-10-thia-5,8-diaza-benzo[a]azulen-9-one (CC2);

7-Benzyl-3-methoxy-5-(3-methoxy-benzyl)-5,6,7,8-tetrahydro-10-thia-5,8-diaza-benzo[a]azulen-9-one (DD2);

3-Methoxy-5-(3-methoxy-benzyl)-7-methyl-5,6,7,8-tetrahydro-10-thia-5,8-diaza-benzo[a]azulen-9-one (EE2);

7-Isopropyl-3-methoxy-5-propyl-5,6,7,8-tetrahydro-10-thia-5,8-diaza-benzo[a]azulen-9-one (FF2);

7-Isopropyl-3-methoxy-5-(3-methoxy-benzyl)-5,6,7,8-tetrahydro-10-thia-5,8-diaza-benzo[a]azulen-9-one (GG2);

3-Methoxy-5-(4-methoxy-benzyl)-7,7 dimethyl-5,6,7,8-tetrahydro-10-thia-5,8-diaza-benzo[a]azulen-9-one (K);

3-Methoxy-7,7-dimethyl-5-propyl-5,6,7,8-tetrahydro-10-thia-5,8-diaza-benzo[a]azulen-9-one (TT);

5-Isobutyl-7,7-dimethyl-5,6,7,8-tetrahydro-10-thia-5,8-diaza-benz[a]azulen-9-one (FF);

5-(2-Methyl-allyl)-5,6,7,8-tetrahydro-10-thia-5,8-diaza-benzo[a]azulen-9-one (PP);

5-(3-Methoxy-benzyl)-7,7-dimethyl-5,6,7,8-tetrahydro-10-thia-5,8-diaza-benz[a]azulen-9-one (QQ);

7,7-Dimethyl-5-(2-methyl-allyl)-5,6,7,8-tetrahydro-10-thia-5,8-diaza-benz[a]azulen-9-one (RR);

5-Isobutyl-7,7-dimethyl-5,6,7,8-tetrahydro-10-thia-5,8-diaza-benz[a]azulen-9-one (SS);

3-Methoxy-7,7-dimethyl-5-(2-methyl-allyl)-5,6,7,8-tetrahydro-10-thia-5,8-diaza-benzo[a]azulen-9-one (UU);

5-(2-Methyl-allyl)-3-nitro-5,6,7,8-tetrahydro-10-thia-5,8-diaza-benz[a]azulen-9-one (Q);

5-propyl-3-nitro-5,6,7,8-tetrahydro-10-thia-5,8-diaza-benz[a]azulen-9-one (S);

5-Isobutyl-3-nitro-5,6,7,8-tetrahydro-10-thia-5,8-diaza-benz[a]azulen-9-one (T);

5-Allyl-3-nitro-5,6,7,8-tetrahydro-10-thia-5,8-diaza-benz[a]azulen-9-one (U);

5-(3-Methoxy-benzyl)-3-nitro-5,6,7,8-tetrahydro-10-thia-5,8-diaza-benz[a]azulen-9-one (V);

5-(3-Methoxy-benzyl)-3-nitro-5,6,7,8-tetrahydro-10-thia-5,8-diaza-benz[a]azulen-9-one (W);

5-Benzyl-3-nitro-5,6,7,8-tetrahydro-10-thia-5,8-diaza-benz[a]azulen-9-one (X);

5-(3-Chloro-benzyl)-3-nitro-5,6,7,8-tetrahydro-10-thia-5,8-diaza-benz[a]azulen-9-one (Y);

5-Ethyl-3-nitro-5,6,7,8-tetrahydro-10-thia-5,8-diaza-benz[a]azulen-9-one (BB);

5-(3-Methyl-but-2-enyl)-3-nitro-5,6,7,8-tetrahydro-10-oxa-5,8-diaza-benz[a]azulen-9-one (CC);

5-(2-Methyl-allyl)-3-nitro-5,6,7,8-tetrahydro-10-oxa-5,8-diaza-benz[a]azulen-9-one (EE);

5-(2-Fluoro-benzyl)-3-nitro-5,6,7,8-tetrahydro-10-thia-5,8-diaza-benz[a]azulen-9-one (GG);

5-n-Butyl-3-nitro-5,6,7,8-tetrahydro-10-thia-5,8-diaza-benz[a]azulen-9-one (DD);

5-Ethyl-3-methoxy-5,6,7,8-tetrahydro-10-oxa-5,8-diaza-benzo[a]azulen-9-one (H);

3-Methoxy-5-(2-methyl-allyl)-5,6,7,8-tetrahydro-10-oxa-5,8-diaza-benzo[a]azulen-9-one (1);

5-Isobutyl-3-methoxy-5,6,7,8-tetrahydro-10-oxa-5,8-diaza-benzo[a]azulen-9-one (N);

3-Chloro-5-propyl-4b,5,6,7,8,9a-hexahydro-10-oxa-5,8-diaza-benzo[a]azulen-9-one (XXI);

5-Butyl-3-chloro-4b,5,6,7,8,9a-hexahydro-10-oxa-5,8-diaza-benzo[a]azulen-9-one (YY4);

5-Butyl-3-chloro-4b,5,6,7,8,9a-hexahydro-10-oxa-5,8-diaza-benzo[a]azulen-9-one (ZZ4);

3-Bromo-5-(2-methyl-allyl)-5,6,7,8-tetrahydro-10-oxa-5,8-diaza-benz[a]azulen-9-one (BB5);

3-Fluoro-5-(2-methyl-allyl)-5,6,7,8-tetrahydro-10-thia-5,8-diaza-benz[a]azulen-9-one (CC5);

5-Butyl-3-fluoro-5,6,7,8-tetrahydro-10-thia-5,8-iaza-benz[a]azulen-9-one (DD5);

5-Allyl-3-fluoro-5,6,7,8-tetrahydro-10-thia-5,8-diaza-benz[a]azulen-9-one (EE5);

5-Benzyl-3-fluoro-5,6,7,8-tetrahydro-10-thia-5,8-diaza-benz[a]azulen-9-one (FF5);

5-Allyl-3-bromo-5,6,7,8-tetrahydro-10-oxa-5,8-diaza-benz[a]azulen-9-one (GG5);

3-Bromo-5-butyl-5,6,7,8-tetrahydro-10-oxa-5,8-diaza-benz[a]azulen-9-one (HH5);

5-Benzyl-3-bromo-5,6,7,8-tetrahydro-10-oxa-5,8-diaza-benz[a]azulen-9-one (II5);

3-Chloro-5-(2-methyl-allyl)-5,6,7,8-tetrahydro-10-thia-5,8-diaza-benz[a]azulen-9-one (KK5);

5-Benzyl-3-chloro-5,6,7,8-tetrahydro-10-thia-5,8-diaza-benz[a]azulen-9-one (LL5);

5-Butyl-3-chloro-5,6,7,8-tetrahydro-10-thia-5,8-diaza-benz[a]azulen-9-one (MM5);

5-Allyl-3-chloro-5,6,7,8-tetrahydro-10-thia-5,8-diaza-benz[a]azulen-9-one (NN5);

3-Bromo-5-(2-fluoro-benzyl)-5,6,7,8-tetrahydro-10-oxa-5,8-diaza-benz[a]azulen-9-one (005);

3-Bromo-5-(3-fluoro-benzyl)-5,6,7,8-tetrahydro-10-oxa-5,8-diaza-benz[a]azulen-9-one (PP5);

3-Chloro-5-(2-fluoro-benzyl)-5,6,7,8-tetrahydro-10-thia-5,8-diaza-benz[a]azulen-9-one (QQ5);

3-Chloro-5-(3-chloro-benzyl)-5,6,7,8-tetrahydro-10-thia-5,8-diaza-benz[a]azulen-9-one (RR5);

3-Chloro-5-(3-methoxy-benzyl)-5,6,7,8-tetrahydro-10-thia-5,8-diaza-benz[a]azulen-9-one (SS5);

3-Chloro-5-(3-methyl-but-2-enyl)-5,6,7,8-tetrahydro-10-thia-5,8-diaza-benz[a]azulen-9-one (TT5);

5-Benzyl-3-chloro-5,6,7,8-tetrahydro-10-oxa-5,8-diaza-benz[a]azulen-9-one (VV5);

3-Fluoro-53-methyl-but-2-enyl)-5,6,7,8-tetrahydro-10-thia-5,8-diaza-benz[a]azulen-9-one (WW5);

3-Fluoro-5-prop-2-ynyl-5,6,7,8-tetrahydro-10-thia-5,8-diaza-benz[a]azulen-9-one (XX5); and

3-Methoxy-5-(3-methyl-butyl)-5,6,7,8-tetrahydro-10-oxa-5,8-diaza-benzo[a]azulen-9-one (YY5).

2-Bromo-5-methoxy-benzaldehyde or 2-bromo-benzaldehyde can be converted to target compounds using the General Methods below as outlined in Scheme 2.

General Method 2-1

The starting 2-bromo-benzaldehyde and two equivalents of the desired diamine are dissolved in 2-ethoxyethanol (cellosolve), or another suitable solvent and heated to 80° C. to 120° C. under a nitrogen atmosphere. After 1 to 5 hours 1 equivalent of elemental sulfur is added to the reaction maintained at high temperature for 4 to 24 hours. After cooling a precipitate forms, which is filtered and washed with water, then dried under vacuum. The product is suitable to carry on to the next reaction.

Compounds prepared by Method 2-1 include:

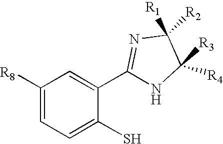

| Example | R₈ | R₁ | R₂ | R₃ | R₄ | Mass Spectra |
|---|---|---|---|---|---|---|
| 12a | OMe | H | H | Methyl | Methyl | m + 1 = 237 |
| 12b | OMe | H | H | H | H | m + 1 = 209 |
| 12c | OMe | Methyl | H | Methyl | H | m + 1 = 237 |
| 12d | H | H | H | Methyl | Methyl | m + 1 = 207 |

Name of compounds:

2-(5,5-Dimethyl-4,5-dihydro-1H-imidazol-2-yl)-4-methoxy-benzenethiol (12a);

2-(4,5-Dihydro-1H-imidazol-2-yl)-4-methoxy-benzenethiol (12b);

2-(4,5-Dimethyl-4,5-dihydro-1H-imidazol-2-yl)-4-methoxy-benzenethiol (12c); and

2-(4,5-Dimethyl-4,5-dihydro-1H-imidazol-2-yl)-benzenethiol (12d).

General Method 2-2

Formation of the benzothiophene is accomplished by reaction with methyl-bromo-acetate as in Step 2 Scheme 2.

Compound 12 is combined with one equivalent of methyl-bromoacetate in methanol and refluxed for 4 to 24 hours under a nitrogen atmosphere. After concentration the product can be isolated by crystallization from chloroform or another suitable solvent, or by column chromatography. The product may be isolated as the HBr salt.

Compounds prepared by this method include:

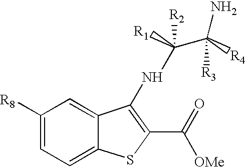

| Example | R₈ | R₁ | R₂ | R₃ | R₄ | Mass Spectra |
|---|---|---|---|---|---|---|
| 13a | OMe | H | H | Methyl | Methyl | M + 1 = 309 |
| 13b | OMe | H | H | H | H | M + 1 = 287 |
| 13c | OMe | Methyl | H | Methyl | H | M + 1 = 309 |
| 13d | H | H | H | Methyl | Methyl | M + 1 = 279 |

Names of compounds:

3-(2-Amino-2-methyl-propylamino)-5-methoxy-benzo[b]thiophene-2-carboxylic acid methyl ester (13a);

3-(2-Amino-ethylamino)-5-methoxy-benzo[b]thiophene-2-carboxylic acid methyl ester (13b);

3-(2-Amino-1-methyl-propylamino)-5-methoxy-benzo[b]thiophene-2-carboxylic-acid methyl ester (13c); and

3-(2-Amino-2-methyl-propylamino)-benzo[b]thiophene-2-carboxylic acid methyl ester (13d).

Compounds are then cyclized using General Method 1-4-1 or 14-2. They can then be derivatized further with General Methods 1-5-1 or 1-5-2.

Compounds from the examples above can be modified as shown in Scheme 3 using the following methods:

General Method 3-(1-2)

When R₈═OMe, Compound A is dissolved in methylene chloride and cooled to 0° C. Boron tribromide (1.1 equivalents) is added dropwise to the solution and the mixture refluxed until complete by thin layer chromatography determination. The reaction is quenched with 1N NaOH and stirred 15 minutes. The precipitate thus formed is filtered and dried to give the intermediate phenol.

This phenol is dissolved in THF under a nitrogen atmosphere and treated with cesium carbonate (1.1 equivalents). After stirring 10 minutes the reaction is treated with 1.1 equivalents alkyl halide, and the reaction refluxed 2 to 78 hours. Addition of more base and alkyl halide may be necessary in some cases to drive the reaction to completion. Upon completion of the reaction, the precipitate is filtered, washed with chloroform, and the combined filtrates concentrated. The product is recrystallized or subjected to column chromatography for purification.

The following compounds were synthesized using these general methods:

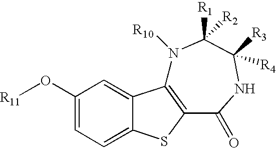

| Example | R₁₀ | R₁₁ | R₁ | R₂ | R₃ | R₄ | Mass Spectra |
|---|---|---|---|---|---|---|---|
| D | n-Propyl | Ethyl | H | H | H | H | (FAB+) m/z = 303.9 |
| E | n-Butyl | Ethyl | H | H | H | H | (FAB) m/z = 319(m + 1) |
| G | i-Butyl | Ethyl | H | H | H | H | (FAB) m/z = 318(M+) |
| M | i-prenyl | Ethyl | H | H | H | H | (FAB+) m/z = 329.9(100), |
| | | | | | | | 331.1(90) (M + H) |
| NN | H | Ethyl | H | H | Me | Me | m + 1 = 291 |

Names of compounds:

3-Ethoxy-5-propyl-5,6,7,8-tetrahydro-10-thia-5,8-diaza-benzo[a]azulen-9-one (D);

5-Butyl-3-ethoxy-5,6,7,8-tetrahydro-10-thia-5,8-diaza-benzo[a]azulen-9-one (E);

3-Ethoxy-5-isobutyl-5,6,7,8-tetrahydro-10-thia-5,8-diaza-benzo[a]azulen-9-one (G);

3-Ethoxy-5-(3-methyl-but-2-enyl)-5,6,7,8-tetrahydro-10-thia-5,8-diaza-benzo[a]azulen-9-one (M); and

3-Ethoxy-7,7-dimethyl-5,6,7,8-tetrahydro-10-thia-5,8-diaza-benzo[a]azulen-9-one (NN).

General Method 4-(1-2)-1

A compound of formula Ib where R₈═H and R₁₀ does not ═H is dissolved in chloroform (EtOH free) and a catalytic amount of DMF, and chilled under an inert atmosphere in an ice bath. To this is added five equivalents ClSO₃H dropwise, and followed by three equivalents thionyl chloride added in a single portion. The reaction is refluxed for 1 to 10 hours, then cooled and ice water added. The organic layer is separated, the aqueous layer extracted with more chloroform, and the combined organics washed with water and brine and dried over a suitable drying agent such as Na₂SO₄. The solution is concentrated and the resulting solid Id used without further purification.

Na₂SO₃ (2 equivalents) and NaHCO₃ (2.1 equivalents) are dissolved in water and the mixture heated to 70° C. to 80° C. To the hot solution is added Id from the previous step in several portions over 1 to 2 hours, maintaining the temperature. The reaction is cooled, stirred for 4 to 24 hours and filtered. The solid thus collected is suspended in water, and treated with a slight excess of dimethylsulfate or another suitable alkylating agent and sodium bicarbonate. The reaction is then refluxed for 10 to 72 hours, cooled and filtered. The solid is washed well with TBF and dried to afford the product Ie.

Compounds synthesized with this method include:

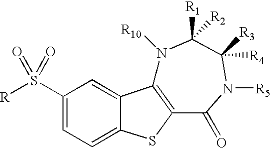

| Example | R | R₁₀ | R₁ | R₂ | R₃ | R₄ | R₅ | Mass Spectra |
|---|---|---|---|---|---|---|---|---|
| RR2 | Me | Isobutyl | H | H | H | H | H | m + 1 = 353 |
| VV2 | Me | Isobutyl | H | H | H | H | Me | m + 1 = 367 |
| XX2 | Me | Isobutyl | H | H | H | H | allyl | m + 1 = 393 |

Name of compounds:

5-Isobutyl-3-methanesulfonyl-5,6,7,8-tetrahydro-10-thia-5,8-diaza-benz[a]azulen-9-one (RR2);

5-Isobutyl-3-methanesulfonyl-8-methyl-5,6,7,8-tetrahydro-10-thia-5,8-diaza-benz[a]azulen-9-one (VV2); and

8-Allyl-5-isobutyl-3-methanesulfonyl-5,6,7,8-tetrahydro-10-thia-5,8-diaza-benz[a]azulen-9-one (XX2).

General Method 4-(1-2)-2

The sodium salt of the sulfinate above is dissolved in water and cooled in an ice bath. Concentrated HCl is used to adjust the pH to 2, and the resulting precipitate filtered. This solid is then dissolved in DMF and potassium carbonate or another suitable base added, followed by an alkylating agent. The reaction is stirred from 1 to 10 hours, then partitioned between water and EtOAc, and the precipitate formed is filtered. This solid is washed with water, methanol, and dried. The solid is then suspended in methanol and 2N NaOH added, and the reaction stirred for 2 to 8 hours. The insoluble material is isolated by filtration and washed with water and methanol. After drying, the solid is recrystallized in THF or another suitable solvent.

Compounds synthesized with this method include:

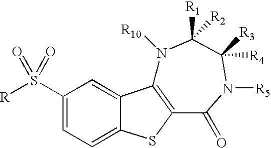

| Example | R | R₁₀ | R₁ | R₂ | R₃ | R₄ | R₅ | Mass Spectra |
|---|---|---|---|---|---|---|---|---|
| UU2 | Allyl | Isobutyl | H | H | H | H | H | m + 1 = 379 |
| WW2 | Benzyl | Isobutyl | H | H | H | H | H | m + 1 = 429 |
| TT2 | Ethyl | Isobutyl | H | H | H | H | H | m + 1 = 367 |

Name of compounds:

5-Isobutyl-3-(prop-2-ene-1-sulfonyl)-5,6,7,8-tetrahydro-10-thia-5,8-diaza-benz[a]azulen-9-one (UU2);

5-Isobutyl-3-phenylmethanesulfonyl-5,6,7,8-tetrahydro-10-thia-5,8-diaza-benz[a]azulen-9-one (WW2); and

3-Ethanesulfonyl-5-isobutyl-5,6,7,8-tetrahydro-10-thia-5,8-diaza-benz[a]azulen-9-one (TT2).

Compounds where R₈ is an electron withdrawing group such as nitro or halogen have been synthesized as outlined in Scheme 5.

General Method 5-(1-3)

The desired amine (1.1 equivalents) 6 is dissolved in an appropriate solvent such as an alcohol or hydrocarbon solvent, under an inert atmosphere to exclude water, and cooled in an ice bath. The appropriate aldehyde 23 (where X═O or S, and EWG is nitro or halogen) is added and the mixture stirred for 0.5 to 4 hours, coming to ambient temperature. The reaction mixture is then concentrated and the resulting imine used in the next step.

The imine 24 is dissolved in DMF and treated with a base such as potassium carbonate (1.1 to 10 equivalents), and methyldichloroacetate (1-1.1 equivalents) while stirring under chilled conditions and under an inert atmosphere. The reaction is allowed to come to ambient temperature over 0.25 to 24 hours. The reaction is re-cooled, then water, hexane, and EtOAc are added, followed by stirring. The reaction is then filtered, and the solid washed with water and hexane. The heterocycle 25 is used as is in the next step.

The BOC-group is removed from the amine by dissolving heterocycle 25 in an appropriate solvent such as dioxane, THF, methylene chloride or the like, under an inert atmosphere, and treating the solution with an acid such as 1N to 4N HCl or TFA. After stirring 15 minutes to 24 hours, the reaction is concentrated. The residue can be treated with several portions toluene, evaporating under reduced pressure between each, to remove excess acid if needed. The resulting amine salt is dissolved in an appropriate solvent such as methanol and treated with base (NaH, sodium metal, or the like, 2-10 equivalents) and the reaction heated for a period of 1 to 24 hours. The reaction is cooled, and the precipitate which develops is collected by filtration. The solid is washed well with water and EtOAc to afford the product. If necessary the filtrate can be subjected to column chromatography to isolate further product.

Compounds prepared by this method include:

| | |
|---|---|
| | Ia |
| 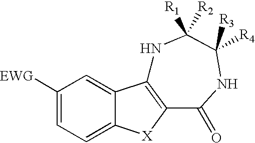 | |

| Example | EWG | X | R₁ | R₂ | R₃ | R₄ | Mass Spectra |
|---|---|---|---|---|---|---|---|
| YY2 | NO₂ | O | H | H | H | H | m + 1 = 248 |
| ZZ2 | NO₂ | O | H | H | H | Me | m + 1 = 262 |
| AA3 | NO₂ | O | H | H | H | iPr | m + 1 = 290 |
| BB3 | NO₂ | O | H | H | Me | H | m + 1 = 262 |
| CC3 | NO₂ | O | H | H | Me | Me | m + 1 = 276 |
| P | NO₂ | S | H | H | H | H | m + 1 = 264 |
| AA5 | Br | O | H | H | H | H | m + 1 = 282 |
| JJ5 | Cl | S | H | H | H | H | m + 1 = 253 |
| UU5 | Cl | O | H | H | H | H | m + 1 = 237 |

Names of compounds:

3-Nitro-5,6,7,8-tetrahydro-10-oxa-5,8-diaza-benz[a]azulen-9-one (YY2);

(S)-7-Methyl-3-nitro-5,6,7,8-tetrahydro-10-oxa-5,8-diaza-benz[a]azulen-9-one (ZZ2);

(S)-7-Isopropyl-3-nitro-5,6,7,8-tetrahydro-10-oxa-5,8-diaza-benz[a]azulen-9-one (AA3);

(R)-7-Methyl-3-nitro-5,6,7,8-tetrahydro-10-oxa-5,8-diaza-benz[a]azulen-9-one (BB3);

7,7-Dimethyl-3-nitro-5,6,7,8-tetrahydro-10-oxa-5,8-diaza-benz[a]azulen-9-one (CC3);

3-Nitro-5,6,7,8-tetrahydro-10-thia-5,8-diaza-benz[a]azulen-9-one (P);

3-Bromo-5,6,7,8-tetrahydro-10-oxa-5,8-diaza-benz[a]azulen-9-one (AA5);

3-Chloro-5,6,7,8-tetrahydro-10-thia-5,8-diaza-benz[a]azulen-9-one (JJ5); and

3-Chloro-5,6,7,8-tetrahydro-10-oxa-5,8-diaza-benz[a]azulen-9-one (UU5).

Compounds from this series can be alkylated to functionalize R₁₀ as described in General Methods 1-5-1 and 1-5-2.

| | |
|---|---|
| | Ia |
| 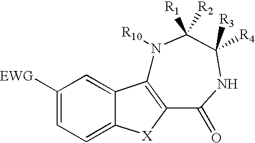 | |

| | | | | | | | | Mass |
|---|---|---|---|---|---|---|---|---|
| Example | EWG | X | R₁₀ | R₁ | R₂ | R₃ | R₄ | Spectra |
| XX4 | Cl | O | n-propyl | H | H | H | H | m + 1 = 279 |
| YY4 | Cl | O | n-butyl | H | H | H | H | m + 1 = 293 |
| ZZ4 | Cl | O | i-pentyl | H | H | H | H | m + 1 = 307 |
| VV5 | Cl | O | Benzyl | H | H | H | H | m + 1 = 327 |
| BB5 | Br | O | 2-methyl-allyl | H | H | H | H | m + 1 = 336 |
| GG5 | Br | O | Allyl | H | H | H | H | m + 1 = 322 |
| HH5 | Br | O | n-butyl | H | H | H | H | m + 1 = 338 |
| II5 | Br | O | Benzyl | H | H | H | H | m + 1 = 372 |
| PP5 | Br | O | 3-fluoro-benzyl | H | H | H | H | m + 1 = 390 |

Names of compounds:

3-Chloro-5-propyl-4b,5,6,7,8,9a-hexahydro-10-oxa-5,8-diaza-benzo[a]azulen-9-one (XX4);

5-Butyl-3-chloro-4b,5,6,7,8,9a-hexahydro-10-oxa-5,8-diaza-benzo[a]azulen-9-one (YY4);

5-Butyl-3-chloro-4b,5,6,7,8,9a-hexahydro-10-oxa-5,8-diaza-benzo[a]azulen-9-one (ZZ4);

5-Benzyl-3-chloro-5,6,7,8-tetrahydro-10-oxa-5,8-diaza-benz[a]azulen-9-one (VV5);

3-Bromo-5-(2-methyl-allyl)-5,6,7,8-tetrahydro-10-oxa-5,8-diaza-benz[a]azulen-9-one (BB5);

5-Allyl-3-bromo-5,6,7,8-tetrahydro-10-oxa-5,8-diaza-benz[a]azulen-9-one (GG5);

3-Bromo-5-butyl-5,6,7,8-tetrahydro-10-oxa-5,8-diaza-benz[a]azulen-9-one (HH5);

5-Benzyl-3-bromo-5,6,7,8-tetrahydro-10-oxa-5,8-diaza-benz[a]azulen-9-one (II5); and

3-Bromo-5-(3-fluoro-benzyl)-5,6,7,8-tetrahydro-10-oxa-5,8-diaza-benz[a]azulen-9-one (PP5).

General Method 5-4

Nitro compounds such as those described can be reduced by catalytic hydrogenation by dissolving the compound in TBF, methanol, DMF, or another suitable solvent in the presence of Raney nickel and a hydrogen atmosphere, which may or may not be pressurized. An acid such as acetic acid may be present. The reaction is stirred vigorously for 0.5 to 10 hours, then filtered, and the filtrate concentrated. The residue can be taken up in methanol and reconcentrated as needed to remove excess acid. Purification by column chromatography yields the final product.

Compounds where R₈ is an electron donating group such as methoxy and the core ring structure is a benzofuran have been synthesized as outlined in Scheme 6.

General Method 6-(1-3)

The desired amine (1.1 equivalents) 6 is dissolved in an appropriate solvent such as an alcohol or hydrocarbon solvent, under an inert atmosphere to exclude water, and cooled in an ice bath. The appropriate aldehyde 26 is added and the mixture stirred for 0.5 to 4 hours, coming to ambient temperature. The reaction mixture is then concentrated and the resulting imine used in the next step.

The imine 27 is dissolved in DMF and treated with a base such as potassium carbonate (1.1 to 10 equivalents), and methyldichloroacetate (1 to 1.1 equivalents) while stirring under chilled conditions and under an inert atmosphere. The reaction is allowed to come to ambient temperature over 0.25 to 24 hours. The reaction is re-cooled, then water, hexane, and EtOAc are added, followed by stirring. The reaction is then filtered, and the solid washed with water and hexane. The cyclized heterocycle 28 is used as is in the next step.

The BOC-group is removed from the amine by dissolving heterocycle 28 in an appropriate solvent such as dioxane, THF, methylene chloride or the like, under an inert atmosphere, and treating the solution with an acid such as 1N to 4N HCl or TFA. After stirring 15 minutes to 24 hours, the reaction is concentrated. The residue can be treated with several portions toluene, evaporating under reduced pressure between each, to remove excess acid if needed. The resulting amine salt is dissolved in an appropriate solvent such as methanol and treated with base (NaH, sodium metal, or the like, 2-10 equivalents) and the reaction heated for a period of 1 to 24 hours. The reaction is cooled, and the precipitate which develops is collected by filtration. The solid is washed well with water and EtOAc to afford the product. If necessary the filtrate can be subjected to column chromatography to isolate further product.

Compounds prepared by this method include:

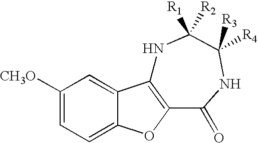

| Example | | | | | Mass Spectra |
|---|---|---|---|---|---|
| No. | R₁ | R₂ | R₃ | R₄ | (m + 1) |
| TT4 | H | H | H | H | 233 |

Name of compounds:

3-Methoxy-5,6,7,8-tetrahydro-10-oxa-5,8-diaza-benzo[a]azulen-9-one (TT4).

Compounds from these examples can be alkylated using methods 1-5-1 and 1-5-2 to give target compounds.

Acylated compounds are prepared as illustrated in Scheme 9.

General Method 7-1-1. Preparation of Amides via BOC Anhydride Activation (Tet. Lett. 1995;36:7115)

A mixture of the appropriate amino acid (1 equivalent) and pyridine (1.1 equivalent) are combined in dioxane in an ice bath for 15 minutes under a nitrogen atmosphere. To this is added in a single portion 1.3 equivalents of BOC-anhydride and the mixture stirred for another 15 minutes, then treated with 1.25 equivalents of ammonium carbonate. The reaction is allowed to come to ambient temperature over 18 hours, then concentrated. The residue is partitioned between EtOAc and H₂O, the organics washed with 1.0N HCl, then H₂O, and brine, dried (Na₂SO₄), and concentrated. The crude reaction is sufficiently pure to use in the next step after vacuum drying at 78° C.

General Method 7-1-2. Preparation of Amides via Ethyl Chloroformate

A mixture of the starting amino acid (1 equivalent) is dissolved in dry THF and chilled in an ice bath under a nitrogen atmosphere. To this is added 1.05 equivalents triethylamine, followed by 1.1 equivalents of the alkylchoro-formate. The mixture is allowed to stir 1 hour while a saturated solution of ammonia/THF is prepared separately. An excess amount of this ammonia solution is added to the activated ester solution, and the mixture allowed to come to ambient temperature over 16 hours. The reaction is then concentrated, the residue partitioned between H₂O and EtOAc, the organic layer separated and washed sequentially with saturated aqueous bicarbonate, H₂O, and brine, dried (Na₂SO₄), and concentrated. The crude reaction is sufficiently pure to use in the next step after vacuum drying at 78° C.

General Method 7-1-3 Preparation of Amides via CDI

A mixture of the starting amino acid (1 equivalent) is dissolved in acetonitrile under a nitrogen atmosphere, and 1.2 equivalents carbonyldiimidazole (CDI) is added. The mixture is refluxed for 1 hour, cooled, and quenched with an excess of ammonium hydroxide (28% aqueous solution). This solution is heated at reflux for 15 minutes, cooled, partially concentrated under reduced pressure, then poured into water. The resulting precipitate is collected, washed twice with water, and dried under vacuum to afford the amide in purity sufficient for the next reaction.

The following compounds were prepared by these general methods:

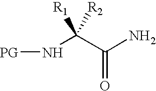

| Example | | | | | Mass Spec |
|---|---|---|---|---|---|
| No. | R₁ | R₂ | PG | Method | (m + 1) |
| 8a | Methyl | H | BOC | 2-1-1 | 189 |
| 8b | H | Methyl | BOC | 2-1-1 | 189 |
| 8c | Benzyl | H | BOC | 2-1-2 | 265 |
| 8d | H | Benzyl | BOC | 2-1-2 | 265 |
| 8e | Methyl | H | CBZ | 2-1-1 | 209 |
| 8f | H | Methyl | CBZ | 2-1-1 | 209 |
| 8g | i-Propyl | H | CBZ | 2-1-3 | 237 |
| 8h | H | i-Propyl | CBZ | 2-1-3 | 237 |
| 8I | Benzyl | H | CBZ | 2-1-2 | 299 |
| 8j | H | Benzyl | CBZ | 2-1-2 | 299 |
| 8k | i-Propyl | H | BOC | 2-1-2 | 218 |
| 8l | H | i-Propyl | BOC | 2-1-2 | 218 |

Names of compounds:

(S)-3-(t-butyloxycarboxy)amino-2-methyl-1-carboxamide (8a);

(R)-3-(t-butyloxycarboxy)amino-2-methyl-1-carboxamide (8b);

(S)-3-(t-butyloxycarboxy)amino-2-benzyl-1-carboxamide (8c);

(R)-3-(t-butyloxycarboxy)amino-2-benzyl-1-carboxamide (8d);

(S)-3-(carbonylbenzyloxy)amino-2-methyl-1-carboxamide (8e);

(R)-3-(carbonylbenzyloxy)amino-2-methyl-1-carboxamide (8f);

(S)-3-(carbonylbenzyloxy)amino-2-iso-propyl-1-carboxamide (8g);

(R)-3-(carbonylbenzyloxy)amino-2-iso-propyl-1-carboxamide (8h);

(S)-3-(carbonylbenzyloxy)amino-2-benzyl-1-carboxamide (8i);

(R)-3-(carbonylbenzyloxy)amino-2-benzyl-1-carboxamide (8j);

(S)-3-(t-butyloxycarboxy)amino-2-iso-propyl-1-carboxamide (8k); and

(R)-3-(t-butyloxycarboxy)amino-2-iso-propyl-1-carboxamide (8l).

General Method 7-2

The amino-amide compound 8 is dissolved in a small amount of DMF under a nitrogen atmosphere and is stirred at room temperature. One-half equivalent of cyanuric chloride is added, the reaction stirred from 5 to 30 minutes, then quenched with water. After stirring an additional 10 to 30 minutes the white solid is filtered, washed with water and saturated aqueous sodium bicarbonate solution, then dried under vacuum. The resulting product is sufficiently pure to use in the next reaction.

The following compounds were made by this method:

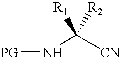

| Example | | | | Mass Spec |
|---|---|---|---|---|
| No. | R₁ | R₂ | PG | (m + 1) |
| 9a | Methyl | H | BOC | 171 |
| 9b | H | Methyl | BOC | 171 |
| 9c | Benzyl | H | BOC | 247 |
| 9d | H | Benzyl | BOC | 247 |
| 9e | Methyl | H | CBZ | 191 |
| 9f | H | Methyl | CBZ | 191 |
| 9g | i-Propyl | H | CBZ | 219 |
| 9h | H | i-Propyl | CBZ | 219 |
| 9I | Benzyl | H | CBZ | 281 |
| 9j | H | Benzyl | CBZ | 281 |
| 9k | i-propyl | H | BOC | 200 |
| 91 | H | i-propyl | BOC | 200 |

Names of compounds:

(S)-(Cyano-methyl-methyl)-carbamic acid tert-butyl ester (9a);

(R)-(Cyano-methyl-methyl)-carbamic acid tert-butyl ester (9b);

(S)-Cyano-methyl-benzyl)-carbamic acid tert-butyl ester (9c);

(R)(Cyano-methyl-benzyl)-carbamic acid tert-butyl ester (9d);

(S)-(Cyano-methyl-methyl)-carbamic acid benzyl ester (9e);

(R)-(Cyano-methyl-methyl)-carbamic acid benzyl ester (9f);

(S)-(1-Cyano-2-methyl-propyl)-carbamic acid benzyl ester (9g);

(R)-(1-Cyano-2-methyl-propyl)-carbamic acid benzyl ester (9h);

(S)-(Benzyl-cyano-methyl)-carbamic acid benzyl ester (9i);

(R)-(Benzyl-cyano-methyl)-carbamic acid benzyl ester (9j);

(S)-(Cyano-methyl-iso-propyl)-carbamic acid tert-butyl ester (9k); and

(R)-(Cyano-methyl-iso-propyl)-carbamic acid tert-butyl ester (9l).

General Method 7-3

The cyano compounds from Step 2 above can be dissolved in freshly distilled THF under a nitrogen atmosphere, and cooled in a water/ice bath. 1.1 to 5.0 equivalents of 1.0 M borane-tetrahydrofuran complex is added dropwise. After 1 hour to overnight of stirring, during which time the reaction may be heated to reflux if needed, the reaction is quenched with methanol, and concentrated. More methanol (ca 5 mL) is added, the reaction re-concentrated, and this procedure repeated ×3. The clear colorless syrup is dried under vacuum for several hours prior to use.

The following compounds were prepared by this method:

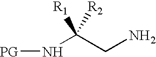

| Example | | | | Mass Spec |
|---|---|---|---|---|
| No. | R₁ | R₂ | PG | (m + 1) |
| 6a-a | Methyl | H | BOC | 175 |
| 6a-b | H | Methyl | BOC | 175 |
| 6a-c | Benzyl | H | BOC | 269 |
| 6a-d | H | Benzyl | BOC | 269 |
| 6a-e | Methyl | H | CBZ | 213 |
| 6a-f | H | Methyl | CBZ | 213 |
| 6a-g | i-Propyl | H | CBZ | 241 |
| 6a-h | H | i-Propyl | CBZ | 241 |
| 6a-i | Benzyl | H | CBZ | 285 |
| 6a-j | H | Benzyl | CBZ | 285 |
| 6a-k | i-Propyl | H | BOC | 204 |
| 6a-l | H | i-Propyl | BOC | 204 |

Names of products:

(S)-(2-Amino-1-methyl-ethyl)-carbamic acid tert-butyl ester (6a-a);

(R)-(2-Amino-1-methyl-ethyl)-carbamic acid tert-butyl ester (6a-b);

(S)-(2-Amino-1-benzyl-ethyl)-carbamic acid tert-butyl ester (6a-c);

(R)-(2-Amino-1-benzyl-ethyl)-carbamic acid tert-butyl ester (6a-d);

(S)-(2-Amino-1-methyl-ethyl)-carbamic acid benzyl ester (6a-e);

(R)-(2-Amino-1-methyl-ethyl)-carbamic acid benzyl ester (6a-f);

(S)-(1-Aminomethyl-2-methyl-propyl)-carbamic acid benzyl ester (6a-g);

(R)-(1-Aminomethyl-2-methylpropyl)-carbamic acid benzyl ester (6a-h);

(S)-(2-Amino-1-benzyl-ethyl)-carbamic acid benzyl ester (6a-i);

(R)-(2-Amino-1-benzyl-ethyl)-carbamic acid benzyl ester (6a-j);

(S)-(2-Amino-1-iso-propyl-ethyl)-carbamic acid tert-butyl ester (6a-k); and

(R)-2-Amino-1-iso-propyl-ethyl)-carbamic acid tert-butyl ester (6a-l).

The amines 6a a-d and 6a k-l can be used in Schemes 1 and 3. They can be reacted further to reverse the protected nitrogen as described below, to afford compounds of structure 6b and thereby substitution in the R₃ and R₄ positions.

General Method 74

An alternative protecting group is introduced on the free amine as follows: The mono-protected diamine from above (6a) is dissolved in dioxane or other appropriate solvent. The reaction is placed under a nitrogen atmosphere at 0° C. to room temperature and to the solution is added one equivalent triethylamine or another non-nucleophilic base, followed by the activated form of the new protecting group, such as BOC-anhydride, CBZ-Cl or the like. The new protecting group is different than the original protecting group in order to allow selective deprotection. The activated protecting group is added dropwise in a solution of the chosen solvent. After stirring for 1 to 4 hours, TLC is taken and if the reaction is incomplete it is heated for 2 to 24 hours at 35° C. to 80° C. After completion the reaction is concentrated, the partitioned between water and EtOAc, the organics separated and washed with brine, dried over sodium sulfate or magnesium sulfate and concentrated. The residue can be recrystallized in a suitable solvent system such as 1:1 hexanes/EtOAc to provide the bis-protected diamine.

The following compounds were made using this general method:

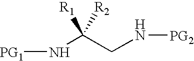

| Example | R₁ | R₂ | PG₁ | PG₂ | Mass Spectra |
|---|---|---|---|---|---|
| 10e | Methyl | H | CBZ | BOC | m + 1 = 309 |
| | | | | | -BOC m + 1 = 209 |
| 10f | H | Methyl | CBZ | BOC | m + 1 = 309 |
| | | | | | -BOC m + 1 = 209 |
| 10g | i-Propyl | H | CBZ | BOC | m + 1 = 337 |
| | | | | | -BOC m + 1 = 237 |
| 10h | H | i-Propyl | CBZ | BOC | m + 1 = 337 |
| | | | | | -BOC m + 1 = 237 |
| 10i | Benzyl | H | CBZ | BOC | m + 1 = 385 |
| | | | | | -BOC m + 1 = 285 |
| 10j | H | Benzyl | CBZ | BOC | m + 1 = 385 |
| | | | | | -BOC m + 1 = 285 |

Name of compounds

(S)-(2-Benzyloxycarbonylamino-propyl)carbamic acid tert-butyl ester (10e);

(R)-(2-Benzyloxycarbonylamino-propyl)-carbamic acid tert-butyl ester (10f);

(S)-(2-Benzyloxycarbonylamino-3-methyl-butyl)-carbamic acid tert-butyl ester (10g);

(R)-(2-Benzyloxycarbonylamino-3-methyl-butyl) carbamic acid tert-butyl ester (10h);

(S)-(2-Benzyloxycarbonylamino-3-phenyl-propyl)-carbamic acid tert-butyl ester (10i); and

(R)-(2-Benzyloxycarbonylamino-3-phenyl-propyl)-carbamic acid tert-butyl ester (10j);

General Method 7-5

Scheme 7 Step 5. The initial protecting group can then be removed to free up the nitrogen adjacent to the α-carbon, thus reversing the regiochemistry of the substitution pattern.

The removal of the CBZ protecting group can be conducted by dissolving the bis-amino compound 10 in a suitable solvent such as THF, addition of a catalyst most commonly 20% palladium on carbon, and subjecting the reaction to a hydrogen atmosphere under pressure. The product is isolated by filtering the reaction through a Celite pad to remove catalyst, concentration of the filtrate, and column chromatography to purify the product.

The following compounds were made with this general method:

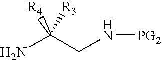

| | | | | Mass Spectra |
|---|---|---|---|---|
| Example | R₃ | R₄ | PG₂ | (m + 1) |
| 6b-a | H | Methyl | BOC | 175 |
| 6b-b | Methyl | H | BOC | 175 |
| 6b-c | H | i-Propyl | BOC | 203 |
| 6b-d | i-Propyl | H | BOC | 203 |
| 6b-e | H | Benzyl | BOC | 251 |
| 6b-f | Benzyl | H | BOC | 251 |

Name of products:

(S)-2-Amino-propyl)-carbamic acid tert-butyl ester (6b-a);

(R)-(2-Amino-propyl)-carbamic acid tert-butyl ester (6b-b);

(S)-(2-Amino-3-methyl-butyl)carbamic acid tert-butyl ester (6b-c);

(R)-(2-Amino-3-methyl-butyl)-carbamic acid tert-butyl ester (6b-d)

(S)-(2-Amino-3-phenyl-propyl)-carbamic acid tert-butyl ester (6b-e); and

(R)-(2-Amino-3-phenyl-propyl)-carbamic acid tert-butyl ester (6b-f).

The protecting group is removed from the nitrogen to afford the free amine. If the protecting group is CBZ, it can be removed under a hydrogen atmosphere with a suitable catalyst. More often it is a BOC functionality, which can be removed under a variety of conditions as described in Greene and Wuts (above), with acidic conditions such as provided by TFA or HCl being successful.

Compounds 6 can be used in Schemes 1 and 2 to provide substitution on the diazepine ring.

Diamine compounds from Scheme 7 can be used in Scheme 2 to afford compounds of structure Formula I which can be further elaborated with the General Methods 1-5-1 and 1-5-2. These compounds can also be elaborated further with General Methods 3-(1-2) if desired. The diamines can be mono-protected, or in the case of the commercially available ethylene diamine or 1,2-diamino-2-methyl-propane used without protection on the nitrogens.

Compounds from the sequences can be elaborated as described in General Methods 3-(1-2).

Introduction of sulfur for R₈ can be conducted as illustrated in Scheme 4.

General Method 8-1

Cyclized compound Ia or Ib is dissolved in a suitable solvent such as THF and treated with 0.9 to 1.5 equivalents of a base such as sodium hydride, potassium bis(trimethylsilyl)amide, or the like, followed by an alkylating agent such as methyl iodide, all under an inert atmosphere. After stirring 15 minutes to 24 hours, the reaction is quenched by pouring into water and extracted with an organic solvent such as EtOAc. The organic layers are washed with brine, dried (MgSO₄ or Na₂SO₄) and concentrated. The product can be tritrated with diethyl ether or purified by column chromatography.

The following compounds were synthesized by this method:

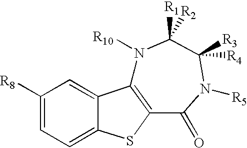

| Ex- | | | | | | | | |
|---|---|---|---|---|---|---|---|---|
| ample | | | | | | | | Mass Spectra |
| No. | R₈ | R₁₀ | R₁ | R₂ | R₃ | R₄ | R5 | (m + 1) |
| J | OMe | 2-methyl- | H | H | H | H | methyl | 317 |
| | | allyl | | | | | | |
| L | OMe | Allyl | H | H | H | H | methyl | 303 |
| 1I4 | OMe | Methyl | H | H | H | H | methyl | 277 |
| KK4 | OMe | 2-methyl- | H | H | H | H | 2-methyl- | 357 |
| | | allyl | | | | | allyl | |
| MM4 | OMe | Propyl | H | H | H | H | methyl | 305 |
| NN4 | Me | i-butyl | H | H | H | H | methyl | 319 |
| OO4 | OMe | 2-methyl- | H | H | H | H | ethyl | 331 |
| | | allyl | | | | | | |
| PP4 | OMe | 2-methyl- | H | H | H | H | allyl | 345 |
| | | allyl | | | | | | |
| QQ4 | OMe | Allyl | H | H | H | H | allyl | 329 |

Names of compounds:

3-Methoxy-8-methyl-5-(2-methyl-allyl)-5,6,7,8-tetrahydro-10-thia-5,8-diaza-benzo[a]azulen-9-one (J);

5-Allyl-3-methoxy-8-methyl-5,6,7,8-tetrahydro-10-thia-5,8-diaza-benzo[a]azulen-9-one (L);

3-Methoxy-5,8-dimethyl-5,6,7,8-tetrahydro-10-thia-5,8-diaza-benzo[a]azulen-9-one (II4);

3-Methoxy-5,8-bis-(2-methyl-allyl}5,6,7,8-tetrahydro-10-thia-5,8-diaza-benzo[a]azulen-9-one (KK4);

3-Methoxy-8-methyl-5-propyl-5,6,7,8-tetrahydro-10-thia-5,8-diaza-benzo[a]azulen-9-one (MM4);

5-Isobutyl-3-methoxy-8-methyl-5,6,7,8-tetrahydro-10-thia-5,8-diaza-benzo[a]azulen-9-one (NN4);

8-Ethyl-3-methoxy-5-(2-methyl-allyl)-5,6,7,8-tetrahydro-10-thia-5,8-diaza-benz[a]azulen-9-one (OO4);

8-Allyl-3-methoxy-5-(2-methyl-allyl)-5,6,7,8-tetrahydro-10-thia-5,8-diaza-benz[a]azulen-9-one (PP4); and

5,8-Diallyl-3-methoxy-5,6,7,8-tetrahydro-10-thia-5,8-diaza-benz[a]azulen-9-one (QQ4).

### Preparation of Diamines (Compounds 6a and b, Schemes 1, 2, 5, 6, 10, and 11, Synthesis Shown in Scheme 7)

Amino acids such as phenyl alanine, alanine, valine, leucine, and the like are commercially available in stereospecific form and can be purchased with a protecting group such as BOC or CBZ in place on the amino functionality. 1,2-Diamino-2-methylpropane is commercially available.

General Method 9-1

The cyclized compound is treated with 1 to 10 equivalents of an acylating agent such as an acid chloride, an anhydride, a mixed anhydride, a chloroformate or the like, usually in the presence of a base such as triethylamine, pyridine, or DMAP. The regio-isomers can be separated as necessary by column chromatography.

Compounds made include:

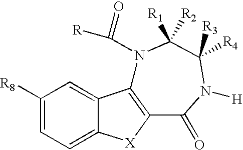

| Example | R₈ | R | X | R₁ | R₂ | R₃ | R4 | Mass Spectra |
|---|---|---|---|---|---|---|---|---|
| GG3 | OCH3 | CH3 | S | H | H | H | H | m + 1 = 291 |

Name of compounds:

5-Acetyl-3-methoxy-5,6,7,8-tetrahydro-10-thia-5,8-diaza-benz[a]azulen-9-one (GG3).

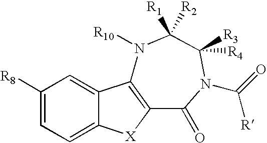

| | | | | | | | | | Mass Spectra |
|---|---|---|---|---|---|---|---|---|---|
| Example | R₈ | R₁₀ | R′ | X | R₁ | R₂ | R₃ | R₄ | (m + 1) |
| UU4 | NO₂ | Benzyl | phenyl | S | H | H | H | H | 458 |
| VV4 | NO₂ | 2-methyl-allyl | phenyl | O | H | H | H | H | 406 |
| WW4 | NO₂ | Allyl | phenyl | S | H | H | H | H | 408 |
| SS4 | OMe | (3-pyridyl)-methyl | methyl | S | H | H | H | H | 382 |

Names of compounds:

8-Benzoyl-5-benzyl-3-nitro-5,6,7,8-tetrahydro-10-thia-5,8-diaza-benz[a]azulen-9-one (UU4);

8-Benzoyl-5-(2-methyl-allyl)-3-nitro-5,6,7,8-tetrahydro-10-oxa-5,8-diaza-benz[a]azulen-9-one (VV4);

5-Allyl-8-benzoyl-3-nitro-5,6,7,8-tetrahydro-10-thia-5,8-diaza-benz[a]azulen-9-one (WW4); and

8-Acetyl-3-methoxy-5-pyridin-3-ylmethyl-5,6,7,8-tetrahydro-10-thia-5,8-diaza-benz[a]azulen-9-one (SS4).

General Method 10-(1-2)

The aldehyde 35 is synthesized from the corresponding methyl compound 33 by first brominating the methyl via treatment with N-bromosuccinimide in the presence of a catalytic amount of a peroxide such as benzoyl peroxide and heating in a halogenated solvent such as carbon tetrachloride for 0.25 to 18 hours. After cooling, the reaction is filtered and the filtrate concentrated. This residue is then dissolved in chloroform or the like and reacted with hexamine. The reaction may be heated for a period of 1 to 10 hours. After cooling the reaction in an ice bath, the resulting crystals are collected. These crystals are dissolved in a 5:1 mixture of acetic acid/water and then heated at 100° C. for 2 to 8 hours. The reaction is cooled to room temperature, then treated with concentrated HCl and heated again at 100° C. for 1 to 3 hours. After cooling, the reaction is extracted with a suitable solvent such as diethyl ether, and the organic layer washed with water, saturated aqueous bicarbonate, water, then dried (Na₂SO₄ or MgSO₄) and concentrated to afford 35.

General Method 10-(3-4)

A stirred solution of 35 in ethoxyethanol or the like is treated dropwise with ethylene diamine 6 (2 equivalents) over 2 to 10 minutes. This is followed by 1 equivalent of elemental sulfur, and the reaction heated to reflux for 2 to 24 hours. After cooling, the reaction is filtered and the solid washed with methanol, and the combined filtrates reduced in volume. To this is added ethyl acetate (with cooling if necessary) to induce crystal formation. These crystals are isolated by filtration to afford 36.

Compound 36 is dissolved in an alcoholic solvent and treated dropwise with methyl bromoacetate (1 equivalent) and then stirred at ambient temperature until reaction is complete. The reaction is concentrated and the residue re-dissolved in DMF and a suitable base such as DBU added. This is then stirred at room temperature to reflux for a period of 0.5 to 18 hours, then cooled if necessary, and water added to the reaction. The precipitate produced is collected and taken up in an organic solvent, washed with brine and dried, and re-concentrated. The solid can be recrystallized from THF/CHCl₃ or the like to afford 1a.

Compounds are alkylated as described in Method 1-5-1 and 1-5-2. Compounds made from these methods include:

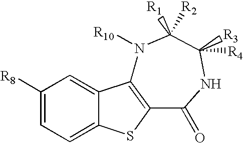

| Example | | | | | | | | Mass Spectra |
|---|---|---|---|---|---|---|---|---|
| No. | R₈ | X | R₁₀ | R₁ | R₂ | R₃ | R₄ | (m + 1) |
| JJ5 | Cl | S | H | H | H | H | H | 253 |
| CC5 | F | S | 2-methyl-allyl | H | H | H | H | 291 |
| DD5 | F | S | n-butyl | H | H | H | H | 293 |
| EE5 | F | S | Allyl | H | H | H | H | 277 |
| FF5 | F | S | Benzyl | H | H | H | H | 327 |
| KK5 | Cl | S | 2-methyl-allyl | H | H | H | H | 307 |
| LL5 | Cl | S | Benzyl | H | H | H | H | 343 |
| MM5 | Cl | S | n-butyl | H | H | H | H | 309 |
| NN5 | Cl | S | Allyl | H | H | H | H | 293 |
| QQ5 | Cl | S | 2-fluoro-benzyl | H | H | H | H | 361 |
| RR5 | Cl | S | 3-chloro-benzyl | H | H | H | H | 378 |
| SS5 | Cl | S | 3-methoxy-benzyl | H | H | H | H | 373 |
| TT5 | Cl | S | iso-pentenyl | H | H | H | H | 321 |
| WW5 | F | S | iso-pentenyl | H | H | H | H | 305 |
| XX5 | F | S | prop-2-ynyl | H | H | H | H | 275 |

Names of compounds

3-Chloro-5,6,7,8-tetrahydro-10-thia-5,8-diaza-benz[a]azulen-9-one (JJ5);

3-Fluoro-5-(2-methyl-allyl)-5,6,7,8-tetrahydro-10-thia-5,8-diaza-benz[a]azulen-9-one (CC5);

5-Butyl-3-fluoro-5,6,7,8-tetrahydro-10-thia-5,8-diaza-benz[a]azulen-9-one (DD5);

5-Allyl-3-fluoro-5,6,7,8-tetrahydro-10-thia-5,8-diaza-benz[a]azulen-9-one (EE5);

5-Benzyl-3-fluoro-5,6,7,8-tetrahydro-10-thia-5,8-diaza-benz[a]azulen 9-one (FF5);

3-Chloro-5-(2-methyl-allyl)-5,6,7,8-tetrahydro-10-thia-5,8-diaza-benz[a]azulen-9-one (KK5);

5-Benzyl-3-chloro-5,6,7,8-tetrahydro-10-thia-5,8-diaza-benz[a]azulen-9-one (LL5);

5-Butyl-3-chloro-5,6,7,8-tetrahydro-10-thia-5,8-diaza-benz[a]azulen-9-one (MM5);

5-Allyl-3-chloro-5,6,7,8-tetrahydro-10-thia-5,8-diaza-benz[a]azulen-9-one (NN5);

3-Chloro-5-(2-fluoro-benzyl)-5,6,7,8-tetrahydro-10-thia-5,8-diaza-benz[a]azulen-9-one (QQ5);

3-Chloro-5-(3-chloro-benzyl)-5,6,7,8-tetrahydro-10-thia-5,8-diaza-benz[a]azulen-9-one (RR5);

3-Chloro-5-(3-methoxy-benzyl)-5,6,7,8-tetrahydro-10-thia-5,8-diaza-benz[a]azulen-9-one (SS5);

3-Chloro-5-(3-methyl-but-2-enyl)-5,6,7,8-tetrahydro-10-thia-5,8-diaza-benz[a]azulen-9-one (TT5);

3-Fluoro-5-(3-methyl-but-2-enyl)-5,6,7,8-tetrahydro-10-thia-5,8-diaza-benz[a]azulen-9-one (WW5); and

3-Fluoro-5-prop-2-ynyl-5,6,7,8-tetrahydro-10-thia-5,8-diaza-benz[a]azulen-9-one (XX5).

An alternative core ring structure useful in these derivatives is the isoquinoline. This is synthesized as shown in Scheme 11 and described in the methods below.

General Method 11-1

Compound 28 is dissolved in distilled THF under an inert atmosphere and cooled in an ice bath. To the solution is added dropwise lithium hexamethyldisilazide (1.2 equivalents) and the reaction stirred 0.25 to 3 hours. The carboxylating agent such as cyanoformate (2.0 equivalents) is added to the reaction, and it is allowed to stir at room temperature for a brief period such as 30 minutes. The reaction is quenched with water, the aqueous layer extracted with EtOAc, and the combined organics washed with brine, dried, and concentrated. The residue is purified by column chromatography to give 29 in 88% yield m+1=206.

General Method 11-(2-3)

Compound 29 is dissolved neat in the diamine 6, such as ethylenediamine, which is used in excess. The reaction is heated to reflux for 15 to 60 minutes then cooled and the excess amine distilled off. Crude 30 is used in the next step.

The amide 30 is dissolved in a solvent such as acetonitrile and the solution refluxed for 2 to 48 hours. After cooling the reaction is filtered and the product 31 collected as a solid. Additional product may be obtained by column chromatography of the filtrate. Combined yield of 77% m+1=215.

General Method 11-(4-5)

Aromatization of the ring is achieved by the following procedure: Compound 31 is suspended in a suitable solvent such as dichloromethane under in inert atmosphere. A suitable base such as triethylamine (2 equivalents) is added, followed by BOC anhydride (4 equivalents) or another appropriate protecting group. Dimethylamiopyridine may be added to enhance reaction rate. The reaction is stirred for 2 to 24 hours, then the solvent removed under reduced pressure. The residue is purified by column chromatography to afford 32. This compound is now more soluble in organic solvents suitable for subsequent reactions.

Compound 32 is combined with 1.1 equivalents N-bromosuccinimide and 0.01 equivalents benzoyl peroxide in carbon tetrachloride. After stirring under reflux for 0.5 to 5 hours, the reaction is cooled and filtered. The filtrate is concentrated to yield the product (85%, m+1=414).

General Method 11-6

The protecting groups are removed from the nitrogens by an appropriate method. In the case of the BOC group, the compound from General Method 10(4-5) is dissolved in an appropriate solvent such as dichloromethane and treated with an excess of an acid such as TFA, HCl, and the like. After stirring for 1 to 24 hours, the solvent is partially removed under vacuum, and diethyl ether added to the reaction which induces formation of a precipitate, which is filtered and dried. This is the acid salt of the product 1k (70%, m=!=214).

Compound made by this method include:

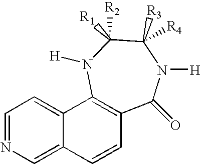

| Example | R₁ | R₂ | R₃ | R₄ | Mass Spectra |
|---|---|---|---|---|---|
| F | H | H | H | H | M + 1 = 214 |

Name of compound:

8,9,10,11-Tetrahydro-3,8,11-triaza-cyclohepta[a]naphthalen-7-one (F).

Compounds made by this method can be reacted further according to General Methods 1-5-1 and 1-5-2 to give compounds such as these:

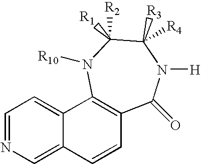

| Example | R₁₀ | R₁ | R₂ | R₃ | R₄ | Mass Spectra |
|---|---|---|---|---|---|---|
| FF4 | 3-methoxy-benzyl | H | H | H | H | m + 1 = 334 |
| GG4 | Methyl | H | H | H | H | m + 1 = 228 |
| HH4 | Ethyl | H | H | H | H | m + 1 = 242 |

Names of compounds:

11-(3-Methoxy-benzyl)-8,9,10,11-tetrahydro-3,8,11-triaza-cyclohepta[a]naphthalen-7-one (FF4);

11-Methyl-8,9,10,11-tetrahydro-3,8,11-triaza-cyclohepta[a]naphthalen-7-one (GG4); and

11-Ethyl-8,9,11-tetrahydro-3,8,11-triaza-cyclohepta[a]naphthalen-7-one (HH4).

1. A compound of Formula I 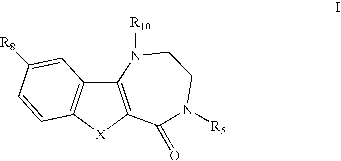
or a pharmaceutically acceptable salt thereof
wherein:
R₈ is H, F, Cl, Br, OR₁₁, NO₂, SO₂R₁₁, N(R₁₁)₂, CN, S—R₁₁ wherein R₁₁ is H, a straight or branched alkyl of 1-6 carbons having from 0 to 1 double or triple bonds, which alkyl is optionally substituted by 0 to 2 groups each independently selected from F, Cl, OR₁₂, and N(R₁₂)₂ wherein R₁₂ is H or straight or branched alkyl of from 1 to 4 carbons which alkyl is optionally substituted by F or OH;
R₁₀ is benzyl unsubstituted or substituted by alkyl, alkoxy, NO₂, halogen, tetrazole, or CN;
R₁₀ is also straight or branched alkyl of from 1 to 4 carbons substituted by 0 to 2 groups each independently selected from F, Cl, OR₁₂, and N(R₁₂)₂ wherein R₁₂ is as described above;
R₁₀ is also straight or branched alkyl of from 2 to 6 carbons having from 0 to 2 double bonds, which alkyl is unsubstituted or substituted; 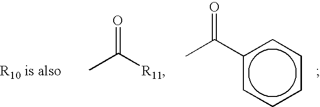 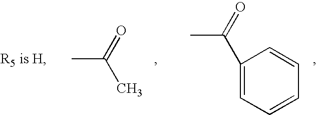
benzyl, or alkyl of from 1 to 4 carbons saturated or unsaturated; and
X is O or S.

2. A compound according to claim 1 wherein R₁₀ is benzyl unsubstituted or substituted by alkyl, alkoxy, NO₂, halogen, tetrazole, or —CN.

3. A compound according to claim 1 wherein R₁₀ is benzyl, unsubstituted or substituted by alkyl, alkoxy, NO₂, halogen, tetrazole, or —CN;
R₈ is halogen, alkoxy, or NO₂;
R₅ is H, 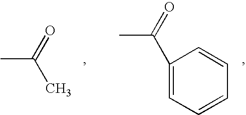
benzyl, or alkyl of from 1 to 4 carbons saturated or unsaturated; and
X is O or S.

4. A compound according to claim 1 and selected from:
3-Methoxy-5-(3-methoxy-benzyl)-5,6,7,8-tetrahydro-10-thia-5,8-diaza-benzo[a]azulen-9-one;
3-Methoxy-5-(4-methoxy-benzyl)-7,7-dimethyl-5,6,7,8-tetrahydro-10-thia-5,8-diaza-benzo[a]azulen-9-one;
3-Methoxy-5-(3-methoxy-benzyl)-7-methyl-5,6,7,8-tetrahydro-10-thia-5,8-diaza-benzo[a]azulen-9-one;
5-(3-Methoxy-benzyl)-3-nitro-5,6,7,8-tetrahydro-10-thia-5,8-diaza-benz[a]azulen-9-one;
5-Benzyl-3-nitro-5,6,7,8-tetrahydro-10-thia-5,8-diaza-benz[a]azulen-9-one;
5-(3-Chloro-benzyl)-3-nitro-5,6,7,8-tetrahydro-10-thia-5,8-diaza-benz[a]azulen-9-one;
5-(2-Fluoro-benzyl)-3-nitro-5,6,7,8-tetrahydro-10-thia-5,8-diaza-benz[a]azulen-9-one;
5-(3-Methoxy-benzyl)-7,7-dimethyl-5,6,7,8-tetrahydro-10-thia-5,8-diaza-benz[a]azulen-9-one;
7-Benzyl-3-methoxy-5-(3-methoxy-benzyl)-5,6,7,8-tetrahydro-10-thia-5,8-diaza-benzo[a]azulen-9-one;
3-Methoxy-5-(3-methoxy-benzyl)-7-methyl-5,6,7,8-tetrahydro-10-thia-5,8-diaza-benzo[a]azulen-9-one;
7-Isopropyl-3-methoxy-5-(3-methoxy-benzyl)-5,6,7,8-tetrahydro-10-thia-5,8-diaza-benzo[a]azulen-9-one;
5-(4-Chloro-benzyl)-3-methoxy-5,6,7,8-tetrahydro-10-thia-5,8-diaza-benzo[a]azulen-9-one;
3-Methoxy-5-(4-methyl-benzyl)-5,6,7,8-tetrahydro-10-thia-5,8-diaza-benzo[a]azulen-9-one;
3-Methoxy-5-(4-methoxy-benzyl)-5,6,7,8-tetrahydro-10-thia-5,8-diaza-benzo[a]azulen-9-one;
5-(2-Chloro-benzyl)-3-methoxy-5,6,7,8-tetrahydro-10-thia-5,8-diaza-benzo[a]azulen-9-one;
3-Methoxy-5-(4-nitro-benzyl)-5,6,7,8-tetrahydro-10-thia-5,8-diaza-benzo[a]azulen-9-one;
3-Methoxy-5-(3-methyl-benzyl)-5,6,7,8-tetrahydro-10-thia-5,8-diaza-benzo[a]azulen-9-one;
5-(3-Chloro-benzyl)-3-methoxy-5,6,7,8-tetrahydro-10-thia-5,8-diaza-benzo[a]azulen-9-one;
3-Methoxy-5-(3-nitro-benzyl)-5,6,7,8-tetrahydro-10-thia-5,8-diaza-benzo[a]azulen-9-one;
3-Methoxy-5-(2-methyl-benzyl)-5,6,7,8-tetrahydro-10-thia-5,8-diaza-benzo[a]azulen-9-one;
3-Methoxy-5-(3-methyl-butyl)-5,6,7,8-tetrahydro-10-thia-5,8-diaza-benz[a]azulen-9-one;
3-Methoxy-5-(2-methoxy-benzyl)-5,6,7,8-tetrahydro-10-thia-5,8-diaza-benz[a]azulen-9-one;
3-Methoxy-5-propyl-5,6,7,8-tetrahydro-10-thia-5,8-diaza-benz[a]azulen-9=one;
4-(3-Methoxy-8-methyl-9-oxo-6,7,8,9-tetrahydro-10-thia-5,8-diaza-benzo[a]azulen-5-ylmethyl)-benzonitrile;
3-Methoxy-5-pyridin-3-ylmethyl-5,6,7,8-tetrahydro-10-thia-5,8-diaza-benz[a]azulen-9-one;
11-(3-Methoxy-benzyl)-8,9,10,11-tetrahydro-3,8,11-triaza-cyclohepta[a]naphthalen-7-one;
8-Acetyl-3-methoxy-S-pyridin-3-ylmethyl-5,6,7,8-tetrahydro-10-thia-5,8-diaza-benz[a]azulen-9-one;
8-Benzoyl-5-benzyl-3-nitro-5,6,7,8-tetrahydro-10-thia-5,8-diaza-benz[a]azulen-9-one;
5-Benzyl-3-fluoro-5,6,7,8-tetrahydro-10-thia-5,8-diaza-benz[a]azulen-9-one;
5-Benzyl-3-bromo-5,6,7,8-tetrahydro-10-oxa-5,8-diaza-benz[a]azulen-9-one;
5-Benzyl-3-chloro-5,6,7,8-tetrahydro-10-thia-5,8-diaza-benz[a]azulen-9-one;
3-Bromo-5-(2-fluoro-benzyl)-5,6,7,8-tetrahydro-10-oxa-5,8-diaza-benz[a]azulen-9-one;
3-Bromo-5-(3-fluoro-benzyl)-5,6,7,8-tetrahydro-10-oxa-5,8-diaza-benz[a]azulen-9-one;
3-Chloro-5-(2-fluoro-benzyl)-5,6,7,8-tetrahydro-10-thia-5,8-diaza-benz[a]azulen-9-one;
3-Chloro-5-(3-chloro-benzyl)-5,6,7,8-tetrahydro-10-thia-5,8-diaza-benz[a]azulen-9-one;
3-Chloro-5-(3-methoxy-benzyl)-5,6,7,8-tetrahydro-10-thia-5,8-diaza-benz[a]azulen-9-one;
5-Benzyl-3-chloro-5,6,7,8-tetrahydro-10-oxa-5,8-diaza-benz[a]azulen-9-one; and
5-Benzyl-3-methoxy-5,6,7,8-tetrahydro-10-thia-5,8-diaza-benzo[a]azulen-9-one.

5. A compound according to claim 1 wherein R₁₀ is straight or branched alkyl of from 1 to 4 carbons substituted by 0 to 2 groups each independently selected from F, Cl, OR₁₂, and N(R₁₂)₂.

6. A compound according to claim 1 wherein R₁₀ is straight or branched alkyl of from 1 to 4 carbons substituted by 0 to 2 groups each independently selected from F, Cl, OR₁₂, and N(R₁₂)₂;
R₈ is halogen, alkoxy, or NO₂;
R₅ is H, 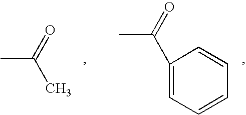
benzyl, or alkyl of from 1 to 4 carbons saturated or unsaturated; and
X is O or S.

7. A compound selected from:
3-(2-tert-Butoxycarbonylamino-ethylamino)-5-methoxy-1-oxo-1H-1λ⁴-benzo[b]thiophene-2-carboxylic acid methyl ester;
(S)-3-(2-tert-Butoxycarbonylamino-propylamino)-5-methoxy-1-oxo-1H-1λ⁴-benzo[b]thiophene-2-carboxylic acid methyl ester;
(R)-3-(2-tert-Butoxycarbonylamino-propylamino)-5-methoxy-1-oxo-1H-1λ⁴-benzo[b]thiophene-2-carboxylic acid methyl ester;
(S)-3-(2-tert-Butoxycarbonylamino-3-phenyl-propylamino)-5-methoxy-1-oxo-1H-1λ⁴-benzo[b]thiophene-2-carboxylic acid methyl ester;
(R)-3-(2-tert-Butoxycarbonylamino-3-phenyl-propylamino)-5-methoxy-1-oxo-1H-1λ⁴-benzo[b]thiophene-2-carboxylic acid methyl ester;
(S)-3-(2-tert-Butoxycarbonylamino-3-methyl-butylamino)-5-methoxy-1-oxo-1H-1λ⁴-benzo[b]thiophene-2-carboxylic acid methyl ester;
(R)-3-(2-tert-Butoxycarbonylamino-3-methyl-butylamino)-5-methoxy-1-oxo-1H-1λ⁴-benzo[b]thiophene-2-carboxylic acid methyl ester;
(S)-3-(2-tert-Butoxycarbonylamino-1-methyl-ethylamino)-5-methoxy-1-oxo-1H-1λ⁴-benzo[b]thiophene-2-carboxylic acid methyl ester;
(R)-3-(2-tert-Butoxycarbonylamino-1-methyl-ethylamino)-5-methoxy-1-oxo-1H-1λ⁴-benzo[b]thiophene-2-carboxylic acid methyl ester;
(S)-3-(1-Benzyl-2-tert-butoxycarbonylamino-ethylamino)-5-methoxy-1-oxo-1H-1λ⁴-benzo[b]thiophene-2-carboxylic acid methyl ester;
(R)-3-(1-Benzyl-2-tert-butoxycarbonylamino-ethylamino)-5-methoxy-1-oxo-1H-1λ⁴-benzo[b]thiophene-2-carboxylic acid methyl ester;
(S)-3-[1-(tert-Butoxycarbonylamino-methyl)-2-methyl-propylamino]-5-methoxy-1-oxo-1H-1λ⁴-benzo[b]thiophene-2-carboxylic acid methyl ester;
(R)-3-[1-(tert-Butoxycarbonylamino-methyl)-2-methyl-propylamino]-5-methoxy-1-oxo-H-1λ⁴-benzo[b]thiophene-2-carboxylic acid methyl ester;
3-(2-Amino-ethylamino)-5-methoxy-1-oxo-1H-1λ⁴-benzo[b]thiophene-2-carboxylic acid methyl ester;
3-(2-tert-Butoxycarbonylamino-ethylamino)-5-methoxy-benzo[b]thiophene-2-carboxylic acid methyl ester;
(S)-3-(2-tert-Butoxycarbonylamino-propylamino)-5-methoxy-benzo[b]thiophene-2-carboxylic acid methyl ester;
(R)-3-(2-tert-Butoxycarbonylamino-propylamino)-5-methoxy-benzo[b]thiophene-2-carboxylic acid methyl ester;
(S)-3-(2-tert-Butoxycarbonylamino-3-phenyl-propylamino 5-methoxy-benzo[b]thiophene-2-carboxylic acid methyl ester;
(R)-3-(2-tert-Butoxycarbonylamino-3-phenyl-propylamino)-5-methoxy-benzo[b]thiophene-2-carboxylic acid methyl ester;
(S)-3-(2-tert-Butoxycarbonylamino-3-methyl-butylamino)-5-methoxy-benzo[b]thiophene-2-carboxylic acid methyl ester;
(R)-3-(2-tert-Butoxycarbonylamino-3-methyl-butylamino)-5-methoxy-benzo[b]thiophene-2-carboxylic acid methyl ester;
(S)-3-(2-tert-Butoxycarbonylamino-1-methyl-ethylamino)-5-methoxy-benzo[b]thiophene-2-carboxylic acid methyl ester;
(R)-3-(2-tert-Butoxycarbonylamino-1-methyl-ethylamino 5-methoxy-benzo[b]thiophene-2-carboxylic acid methyl ester;
(S)-3-(1-Benzyl-2-tert-butoxycarbonylamino-ethylamino)-5-methoxy-benzo[b]thiophene-2-carboxylic acid methyl ester;
(R)-3-(1-Benzyl-2-tert-butoxycarbonylamino-ethylamino)-5-methoxy-benzo[b]thiophene-2-carboxylic acid methyl ester;
(S)-3-[1-(tert-Butoxycarbonylamino-methyl)-2-methyl-propylamino]-methoxy-benzo[b]thiophene-2-carboxylic acid methyl ester;
(R)-3-[1-(tert-Butoxycarbonylamino-methyl)-2-methyl-propylamino]-5-methoxy-benzo[b]thiophene-2-carboxylic acid methyl ester;
3-(2-Amino-ethylamino)-5-methoxy-benzo[b]thiophene-2-carboxylic acid methyl ester;
3-(2-Amino-ethylamino)-5-methoxy-benzo[b]thiophene-2-carboxylic acid methyl ester;
(S)-3-(2-Amino-propylamino)-5-methoxy-benzo[b]thiophene-2-carboxylic acid methyl ester;
(R)-3-(2-Amino-propylamino)-5-methoxy-benzo[b]thiophene-2-carboxylic acid methyl ester;
(S)-3-(2-Amino-3-phenyl-propylamino)-5-methoxy-benzo[b]thiophene-2-carboxylic acid methyl ester;
(R)-3-(2-Amino-3-phenyl-propylamino)-5-methoxy-benzo[b]thiophene-2-carboxylic acid methyl ester;
(S)-3-(2-Amino-3-methyl-butylamino)-5-methoxy-benzo[b]thiophene-2-carboxylic acid methyl ester;
(R)-3-(2-Amino-3-methyl-butylamino)-5-methoxy-benzo[b]thiophene-2-carboxylic acid methyl ester;
(S)-3-(2-Amino-1-methyl-ethylamino)-5-methoxy-benzo[b]thiophene-2-carboxylic acid methyl ester;
(R)-3-(2-Amino-1-methyl-ethylamino)-5-methoxy-benzo[b]thiophene-2-carboxylic acid methyl ester;
(S)-3-(2-Amino-1-benzyl-ethylamino)-5-methoxy-benzo[b]thiophene-2-carboxylic acid methyl ester;
(R)-3-(2-Amino-1-benzyl-ethylamino)-5-methoxy-benzo[b]thiophene-2-carboxylic acid methyl ester;
(S)-3-(1-Aminomethyl-2-methyl-propylamino)-5-methoxy-benzo[b]thiophene-2-carboxylic acid methyl ester;
(R)-3-(1-Aminomethyl-2-methyl-propylamino)-5-methoxy-benzo[b]thiophene-2-carboxylic acid methyl ester;
3-Methoxy-5,6,7,8-tetrahydro-10-thia-5,8-diaza-benzo[a]azulen-9-one;
3-Methoxy-7-methyl-5,6,7,8-tetrahydro-10-thia-5,8-diaza-benzo[a]azulen-9-one (S);
3-Methoxy-7-methyl-5,6,7,8-tetrahydro-10-thia-5,8-diaza-benzo[a]azulen-9-one (R);
7-Benzyl-3-methoxy-5,6,7,8-tetrahydro-10-thia-5,8-diaza-benzo[a]azulen-9-one (S);
7-Benzyl-3-methoxy-5,6,7,8-tetrahydro-10-thia-5,8-diaza-benzo[a]azulen-9-one (R);
7-Isopropyl-3-methoxy-5,6,7,8-tetrahydro-10-thia-5,8-diaza-benzo[a]azulen-9-one (S);
7-Isopropyl-3-methoxy-5,6,7,8-tetrahydro-10-thia-5,8-diaza-benzo[a]azulen-9-one (R);
3-Methoxy-6-methyl-5,6,7,8-tetrahydro-10-thia-5,8-diaza-benzo[a]azulen-9-one (S);
3-Methoxy-6-methyl-5,6,7,8-tetrahydro-10-thia-5,8-diaza-benzo[a]azulen-9-one (R);
6-Benzyl-3-methoxy-5,6,7,8-tetrahydro-10-thia-5,8-diaza-benzo[a]azulen-9-one (S);
6-Benzyl-3-methoxy-5,6,7,8-tetrahydro-10-thia-5,8-diaza-benzo[a]azulen-9-one (R);
3-Methoxy-6-iso-propyl-5,6,7,8-tetrahydro-10-thia-5,8-diaza-benzo[a]azulen-9-one (S);
3-Methoxy-7,7-dimethyl-5,6,7,8-tetrahydro-10-thia-5,8-diaza-benzo[a]azulen-9-one;
2-(5,5-Dimethyl-4,5-dihydro-1H-imidazol-2-yl)-4-methoxy-benzenethiol;
2-(4,5-Dihydro-1H-imidazol-2-yl)-4-methoxy-benzenethiol;
2-(4,5-Dimethyl-4,5-dihydro-1H-imidazol-2-yl)-4-methoxy-benzenethiol;
2-(4,5-Dimethyl-4,5-dihydro-1H-imidazol-2-yl)-benzenethiol;
3-(2-Amino-2-methyl-propylamino)-5-methoxy-benzo[b]thiophene-2-carboxylic acid methyl ester;
3-(2-Amino-ethylamino)-5-methoxy-benzo[b]thiophene-2-carboxylic acid methyl ester;
3-(2-Amino-1-methyl-propylamino)-5-methoxy-benzo[b]thiophene-2-carboxylic acid methyl ester;
3-(2-Amino-2-methyl-propylamino)-benzo[b]thiophene-2-carboxylic acid methyl ester;
5-Isobutyl-3-methanesulfonyl-5,6,7,8-tetrahydro-10-thia-5,8-diaza-benz[a]azulen-9-one;
5-Isobutyl-3-methanesulfonyl-8-methyl-5,6,7,8-tetrahydro-10-thia-5,8-diaza-benz[a]azulen-9-one;
8-Allyl-S-isobutyl-3-methanesulfonyl-5,6,7,8-tetrahydro-10-thia-5,8-diaza-benz[a]azulen-9-one;
3-Nitro-5,6,7,8-tetrahydro-10-oxa-5,8-diaza-benz[a]azulen-9-one;
(S)-7-Methyl-3-nitro-5,6,7,8-tetrahydro-10-oxa-5,8-diaza-benz[a]azulen-9-one;
(S)-7-Isopropyl-3-nitro-5,6,7,8-tetrahydro-10-oxa-5,8-diaza-benz[a]azulen-9-one;
(R)-7-Methyl-3-nitro-5,6,7,8-tetrahydro-10-oxa-5,8-diaza-benz[a]azulen-9-one;
7,7-Dimethyl-3-nitro-5,6,7,8-tetrahydro-10-oxa-5,8-diaza-benz[a]azulen-9-one;
3-Nitro-5,6,7,8-tetrahydro-10-thia-5,8-diaza-benz[a]azulen-9-one;
3-Bromo-5,6,7,8-tetrahydro-10-oxa-5,8-diaza-benz[a]azulen-9-one;
3-Chloro-5,6,7,8-tetrahydro-10-thia-5,8-diaza-benz[a]azulen-9-one;
3-Chloro-5,6,7,8-tetrahydro-10-oxa-5,8-diaza-benz[a]azulen-9-one;
3-Chloro-5-propyl-4b,5,6,7,8,9a-hexahydro-10-oxa-5,8-diaza-benzo[a]azulen-9-one;
5-Butyl-3-chloro-4b,5,6,7,8,9a-hexahydro-10-oxa-5,8-diaza-benzo[a]azulen-9-one;
5-Butyl-3-chloro-4b,5,6,7,8,9a-hexahydro-10-oxa-5,8-diaza-benzo[a]azulen-9-one;
5-Benzyl-3-chloro-5,6,7,8-tetrahydro-10-oxa-5,8-diaza-benz[a]azulen-9-one;
3-Bromo-5-(2-methyl-allyl)-5,6,7,8-tetrahydro-10-oxa-5,8-diaza-benz[a]azulen-9-one;
5-Allyl-3-bromo-5,6,7,8-tetrahydro 10-oxa-5,8-diaza-benz[a]azulen-9-one;
3-Bromo-5-butyl-5,6,7,8-tetrahydro-10-oxa-5,8-diaza-benz[a]azulen-9-one;
5-Benzyl-3-bromo-5,6,7,8-tetrahydro 10-oxa-5,8-diaza-benz[a]azulen-9-one;
3-Bromo-5-(3-fluoro-benzyl)-5,6,7,8-tetrahydro-10-oxa-5,8-diaza-benz[a]azulen-9-one;
3-Methoxy-5,6,7,8-tetrahydro-10-oxa-5,8-diaza-benzo[a]azulen-9-one;
5-Acetyl-3-methoxy-5,6,7,8-tetrahydro-10-thia-5,8-diaza-benzo[a]azulen-9-one;
8-Benzoyl-5-benzyl-3-nitro-5,6,7,8-tetrahydro-10-thia-5,8-diaza-benz[a]azulen-9-one;
8-Benzoyl-5-(2-methyl-allyl)-3-nitro-5,6,7,8-tetrahydro-10-oxa-5,8-diaza-benz[a]azulen-9-one;
5-Allyl-8-benzoyl-3-nitro-5,6,7,8-tetrahydro-10-thia-5,8-diaza-benz[a]azulen-9-one;
8-Acetyl-3-methoxy-5-pyridin-3-ylmethyl-5,6,7,8-tetrahydro-10-thia-5,8-diaza-benz[a]azulen-9-one;
3-Chloro-5,6,7,8-tetrahydro-10-thia-5,8 diaza-benz[a]azulen-9-one;
3-Fluoro-5-(2-methyl-allyl)-5,6,7,8-tetrahydro-10-thia-5,8-fiaza-benz[a]azulen-9-one;
5-Butyl-3-fluoro-5,6,7,8-tetrahydro-10-thia-5,8-diaza-benz[a]azulen-9-one;
5-Allyl-3-fluoro-5,6,7,8-tetrahydro-10-thia-5,8-diaza-benz[a]azulen-9-one;
5-Benzyl-3-fluoro-5,6,7,8-tetrahydro-10-thia-5,8-diaza-benz[a]azulen-9-one;
3-Chloro-5-(2-methyl-allyl)-5,6,7,8-tetrahydro-10-thia-5,8-diaza-benz[a]azulen-9-one;
5-Benzyl-3-chloro-5,6,7,8-tetrahydro-10-thia-5,8-diaza-benz[a]azulen-9-one;
5-Butyl-3-chloro-5,6,7,8-tetrahydro-10-thia-5,8-diaza-benz[a]azulen-9-one;
5-Allyl-3-chloro-5,6,7,8-tetrahydro-10-thia-5,8-diaza-benz[a]azulen-9-one;
3-Chloro-52-fluoro-benzyl)-5,6,7,8-tetrahydro-10-thia-5,8-diaza-benz[a]azulen-9-one;
3-Chloro-5-(3-chloro-benzyl)-5,6,7,8-tetrahydro-10-thia-5,8-diaza-benz[a]azulen-9-one;
3-Chloro-5-(3-methoxy-benzyl)-5,6,7,8-tetrahydro-10-thia-5,8-diaza-benz[a]azulen-9-one;
3-Chloro-5-(3-methyl-but-2-enyl)-5,6,7,8-tetrahydro-10-thia-5,8-diaza-benz[a]azulen-9-one;
3-Fluoro-5-(3-methyl-but-2-enyl)-5,6,7,8-tetrahydro-10-thia-5,8-diaza-benz[a]azulen-9-one; and
3-Fluoro-5-prop-2-ynyl-5,6,7,8-tetrahydro-10-thia-5,8-diaza-benz[a]azulen-9-one.

8. A compound according to claim 1 and selected from:
3-Ethoxy-5-propyl-5,6,7,8-tetrahydro-10-thia-5,8-diaza-benzo[a]azulen-9-one;
5-Butyl-3-ethoxy-5,6,7,8-tetrahydro-10-thia-5,8-diaza-benzo [a]azulen-9-one;
3-Ethoxy-5-isobutyl-5,6,7,8-tetrahydro-10-thia-5,8-diaza-benzo[a]azulen-9-one;
5-Ethyl-3-methoxy-5,6,7,8-tetrahydro-10-oxa-5,8-diaza-benzo[a]azulen-9-one;
3-Methoxy-5-(2-methyl-allyl)-5,6,7,8-tetrahydro-10-oxa-5,8-diaza-benzo[a]azulen-9-one;
3-Methoxy-8-methyl-5-(2-methyl-allyl)-5,6,7,8-tetrahydro-10-thia-5,8-diaza-benzo[a]azulen-9-one;
5-Allyl-3-methoxy-8-methyl-5,6,7,8-tetrahydro-10-thia-5,8-diaza-benzo[a]azulen-9-one;
5-Isobutyl-3-methoxy-5,6,7,8-tetrahydro-10-oxa-5,8-diaza-benzo[a]azulen-9-one;
5-(2-Methyl-allyl)-3-nitro-5,6,7,8-tetrahydro-10-thia-5,8-diaza-benz[a]azulen-9-one;
7-Benzyl-3-methoxy-5-propyl-5,6,7,8-tetrahydro-10-thia-5,8-diaza-benzo[a]azulen-9-one;
5-propyl-3-nitro-5,6,7,8-tetrahydro-10-thia-5,8-diaza-benz[a]azulen-9-one;
5-Isobutyl-3-nitro-5,6,7,8-tetrahydro-10-thia-5,8-diaza-benz[a]azulen-9-one;
5-Allyl-3-nitro-5,6,7,8-tetrahydro-10-thia-5,8-diaza-benz[a]azulen-9-one;
5H-[1]Benzothieno[2,3-f][1,2,4]triazolo[4,3-d][1,4]diazepine,6,7-dihydro-7-(2-methyl-2-propenyl)-9-nitro;
5-Isobutyl-3-methylsulfanyl-5,6,7,8-tetrahydro-10-thia-5,8-diaza-benz[a]azulen-9-one;
5-Ethyl-3-nitro-5,6,7,8-tetrahydro-10-thia-5,8-diaza-benz[a]azulen-9-one;
3-Nitro-5-butyl-5,6,7,8-tetrahydro-10-thia-5,8-diaza-benz[a]azulen-9-one;
5-(2-Methyl-allyl)-3-nitro-5,6,7,8-tetrahydro-10-oxa-5,8-diaza-benz[a]azulen-9-one;
5-Isobutyl-7,7-dimethyl-5,6,7,8-tetrahydro-10-thia-5,8-diaza-benz[a]azulen-9-one;
3-Methoxy-5-(2-methyl-allyl)-5,6,7,8-tetrahydro-10-thia-5,8-diaza-benzo[a]azulen-9-one;
3-Methoxy-5-propyl-5,6,7,8-tetrahydro-10-thia-5,8-diaza-benz[a]azulen-9-one;
7,7-Dimethyl-5-(2-methyl-allyl)-5,6,7,8-tetrahydro-10-thia-5,8-diaza-benz[a]azulen-9-one;
5-Isobutyl-7,7-dimethyl-5,6,7,8-tetrahydro-10-thia-5,8-diaza-benz[a]azulen-9-one;
3-Methoxy-7,7-dimethyl-5-propyl-5,6,7,8-tetrahydro-10-thia-5,8-diaza-benzo [a]azulen-9-one;
3-Methoxy-7,7-dimethyl-5-(2-methyl-allyl)-5,6,7,8-tetrahydro-10-thia-5,8-diaza-benzo[a]azulen-9-one;
3-Methoxy-7-methyl-5-propyl-5,6,7,8-tetrahydro-10-thia-5,8-diaza-benzo[a]azulen-9-one;
3-Methoxy-7-methyl-5-(2-methyl-allyl)-5,6,7,8-tetrahydro-10-thia-5,8-diaza-benzo[a]azulen-9-one;
7-Benzyl-3-methoxy-5-propyl-5,6,7,8-tetrahydro-10-thia-5,8-diaza-benzo[a]azulen-9-one;
7-Benzyl-3-methoxy-5-(2-methyl-allyl)-5,6,7,8-tetrahydro-10-thia-5,8-diaza-benzo[a]azulen-9-one;
7-Benzyl-3-methoxy-5-(2-methyl-allyl)-5,6,7,8-tetrahydro-10-thia-5,8-diaza-benzo[a]azulen-9-one;
7-Isopropyl-3-methoxy-5-propyl-5,6,7,8-tetrahydro-10-thia-5,8-diaza-benzo[a]azulen-9-one;
5-(2-Chloro-allyl)-3-methoxy-5,6,7,8-tetrahydro-10-thia-5,8-diaza-benzo[a]azulen-9-one;
5-Ethyl-3-methoxy-5,6,7,8-tetrahydro-10-thia-5,8-diaza-benz[a]azulen-9-one;
11-Methyl-8,9,10,1-tetrahydro-3,8,11-triaza-cyclohepta[a]naphthalen-7-one;
11-Ethyl-8,9,10,11-tetrahydro-3,8,11-triaza-cyclohepta[a]naphthalen-7-one;
3-Methoxy-5,8-dimethyl-5,6,7,8-tetrahydro-10-thia-5,8-diaza-benzo[a]azulen-9-one;
8-Allyl-3-methoxy-5-(2-methyl-allyl)-5,6,7,8-tetrahydro-10-thia-5,8-diaza-benz[a]azulen-9-one;
5,8-Diallyl-3-methoxy-5,6,7,8-tetrahydro-10-thia-5,8-diaza-benz[a]azulen-9-one;
8-Benzyl-3-methoxy-5-(2-methyl-allyl)-5,6,7,8-tetrahydro-10-thia-5,8-diaza-benz[a]azulen-9-one;
8-Benzoyl-5-(2-methyl-allyl)-3-nitro-5,6,7,8-tetrahydro-10-oxa-5,8-diaza-benz[a]azulen-9-one;
5-Allyl-3-fluoro-5,6,7,8-tetrahydro-10-thia-5,8-diaza-benz[a]azulen-9-one;
5-Allyl-3-bromo-5,6,7,8-tetrahydro-10-oxa-5,8-diaza-benz[a]azulen-9-one;
3-Bromo-5-butyl-5,6,7,8-tetrahydro-10-oxa-5,8-diaza-benz[a]azulen-9-one;
3-Chloro-5-(2-methyl-allyl)-5,6,7,8-tetrahydro-10-thia-5,8-diaza-benz[a]azulen-9-one;
5-Butyl-3-chloro-5,6,7,8-tetrahydro-10-thia-5,8-diaza-benz[a]azulen-9-one;
5-Allyl-3-chloro-5,6,7,8-tetrahydro-10-thia-5,8-diaza-benz[a]azulen-9-one;
3-Fluoro-5-prop-2-ynyl-5,6,7,8-tetrahydro-10-thia-5,8-diaza-benz[a]azulen-9-one;
5-Butyl-3-methoxy-5,6,7,8-tetrahydro-10-thia-5,8-diaza-benz[a]azulen-9-one;
5-Isobutyl-3-methoxy-5,6,7,8-tetrahydro-10-thia-5,8-diaza-benzo[a]azulen-9-one;
5-(2-Methyl-allyl)-5,6,7,8-tetrahydro-10-thia-5,8-diaza-benzo[a]azulen-9-one;
5-Isobutyl-3-methanesulfonyl-5,6,7,8-tetrahydro-10-thia-5,8-diaza-benz[a]azulen-9-one;
3-Ethanesulfonyl-5-isobutyl-5,6,7,8-tetrahydro-10-thia-5,8-diaza-benz[a]azulen-9-one;
5-Isobutyl-3-(prop-2-ene-1-sulfonyl)-5,6,7,8-tetrahydro-10-thia-5,8-diaza-benz[a]azulen-9-one;
5-Isobutyl-3-methanesulfonyl-8-methyl-5,6,7,8-tetrahydro-10-thia-5,8-diaza-benz[a]azulen-9-one;
5-Isobutyl-3-phenylmethanesulfonyl-5,6,7,8-tetrahydro-10-thia-5,8-diaza-benz[a]azulen-9-one;
8-Allyl-5-isobutyl-3-methanesulfonyl-5,6,7,8-tetrahydro-10-thia-5,8-diaza-benz[a]azulen-9-one;
3-Methoxy-5-methyl-5,6,7,8-tetrahydro-10-thia-5,8-diaza-benzo[a]azulen-9-one;
5-Acetyl-3-methoxy-5,6,7,8-tetrahydro-10-thia-5,8-diaza-benzo[a]azulen-9-one;
5-Allyl-3-methoxy-5,6,7,8-tetrahydro 10-thia-5,8-diaza-benzo[a]azulen-9-one;
(3-Methoxy-9-oxo-6,7,8,9-tetrahydro-10-thia-5,8-diaza-benzo[a]azulen-5-yl)-acetic acid;
5-Benzyl-3-methoxy-5,6,7,8-tetrahydro-10-thia-5,8-diaza-benzo[a]azulen-9-one;
3-Methoxy-5,8-bis-(2-methyl-allyl)-5,6,7,8-tetrahydro-10-thia-5,8-diaza-benzo[a]azulen-9-one;
3-Methoxy-8-methyl-5-propyl-5,6,7,8-tetrahydro-10-thia-5,8-diaza-benzo[a]azulen-9-one;
5-Isobutyl-3-methoxy-8-methyl-5,6,7,8-tetrahydro-10-thia-5,8-diaza-benzo[a]azulen-9-one;
8-Ethyl-3-methoxy-5-(2-methyl-allyl)-5,6,7,8-tetrahydro-10-thia-5,8-diaza-benz[a]azulen-9-one;
3-Chloro-5-propyl-4b,5,6,7,8,9a-hexahydro-10-oxa-5,8-diaza-benzo[a]azulen-9-one;
5-Butyl-3-chloro-4b,5,6,7,8,9a-hexahydro-10-oxa-5,8-diaza-benzo[a]azulen-9-one;
3-Bromo-5-(2-methyl-allyl)-5,6,7,8-tetrahydro-10-oxa-5,8-diaza-benz[a]azulen-9-one;
3-Fluoro-5-(2-methyl-allyl)-5,6,7,8-tetrahydro-10-thia-5,8-diaza-benz[a]azulen-9-one; and
5-Butyl-3-fluoro-5,6,7,8-tetrahydro-10-thia-5,8-diaza-benz[a]azulen-9-one.

9. A compound according to claim 1 wherein R₁₀ is straight or branched alkyl of from 2 to 6 carbons having from 0 to 2 double or triple bonds which alkyl is unsubstituted or substituted by halogen or OR₁₂.

10. A compound according to claim 1 wherein R₁₀ is straight or branched alkyl of from 2 to 6 carbons having from 0 to 1 double or triple bonds which alkyl is unsubstituted or substituted by halogen.
R₈ is halogen, alkoxy, or NO₂;
R₅ is H, 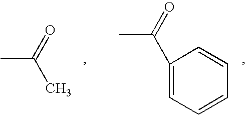
benzyl, or alkyl of from 1 to 4 carbons saturated or unsaturated; and
X is O or S.

11. A compound according to claim 1 and selected from:
3-Ethoxy-5-(3-methyl-but-2-enyl)-5,6,7,8-tetrahydro-10-thia-5,8-diaza-benzo[a]azulen-9-one;
5-(3-Methyl-but-2-enyl)-3-nitro-5,6,7,8-tetrahydro-10-thia-5,8-diaza-benz[a]azulen-9-one;
3-Chloro-5-(3-methyl-but-2-enyl)-5,6,7,8-tetrahydro-10-thia-5,8-diaza-benz[a]azulen-9-one;
3-Fluoro-5-(3-methyl-but-2-enyl)-5,6,7,8-tetrahydro-10-thia-5,8-diaza-benz[a]azulen-9-one;
3-Methoxy-5-(3-methyl-but-2-enyl)-5,6,7,8-tetrahydro-10-thia-5,8-diaza-benzo[a]azulen-9-one;
3-Methoxy-5-(3-methyl-butyl)-5,6,7,8-tetrahydro-10-thia-5,8-diaza-benz[a]azulen-9-one; and
5-Butyl-3-chloro-4b,5,6,7,8,9a-hexahydro-10-oxa-5,8-diaza-benzo[a]azulen-9-one.

12. A method for treating and/or preventing herpes viral infections which comprises administering to a mammal in need of said treatment and/or prevention a compound according to claim 1.

13. A method for treating and/or preventing herpes viral infections which comprises administering to a mammal in need of said treatment and/or prevention a condition caused by herpes simplex I.

14. A method for treating and/or preventing herpes viral infections which comprises administering to a mammal in need of said treatment and/or prevention of cold sores.

15. A method for treating and/or preventing herpes viral infections which comprises administering to a mammal in need of said treatment and/or prevention a condition caused by herpes simplex II.

16. A method for treating and/or preventing herpes viral infections which comprises administering to a mammal in need of said treatment and/or prevention of genital herpes.

17. A method for treating and/or preventing herpes viral infections which comprises administering to a mammal in need of said treatment and/or prevention of shingles caused by herpes zoster.

18. A method for treating and/or preventing herpes viral infections which comprises administering to a mammal in need of said treatment and/or prevention of infections caused by cytomegalovirus (CMV).

19. A method for treating and/or preventing herpes viral infections which comprises administering to a mammal in need of said treatment and/or prevention of Epstein Barr virus.

20. A method for treating and/or preventing herpes viral infections which comprises administering to a mammal in need of said treatment and/or prevention of varicella zoster virus.

21. A method for treating and/or preventing herpes viral infections which comprises administering to a mammal in need of said treatment and/or prevention of pseudorabies.

22. A pharmaceutical composition comprising an amount of a compound of claim 1 effective to treat and/or prevent herpes viral infection in a mammal and a pharmaceutically acceptable carrier.

23. A compound selected from:
8,9,10,11-Tetrahydro-3,8,11-triaza-cyclohepta[a]naphthalen-7-one;
3-Methoxy-5-(3-methoxy-benzyl)-5,6,7,8-tetrahydro-10-thia-5,8-diaza-benzo[a]azulen-9-one;
11-(3-Methoxy-benzyl)-8,9,10,11-tetrahydro-3,8,11-triaza-cyclohepta[a]naphthalen-7-one;
3-Methoxy-5-(3-methoxy-benzyl)-7-methyl-5,6,7,8-tetrahydro-10-thia-5,8-diaza-benzo[a]azulen-9-one; and
5-(3-Methoxy-benzyl)-3-nitro-5,6,7,8-tetrahydro-10-thia-5,8-diaza-benz[a]azulen-9-one.

24. A compound according to claim 1 wherein the compound exhibits improved therapeutic index and improved metabolic stability.

25. A compound according to claim 1 having improved therapeutic index and improved metabolic stability selected from:
3-Ethoxy-5-propyl-5,6,7,8-tetrahydro-10-thia-5,8-diaza-benzo[a]azulen-9-one;
5-Ethyl-3-methoxy-5,6,7,8-tetrahydro-10-oxa-5,8-diaza-benzo[a]azulen-9-one;
3-Methoxy-8-methyl-5-(2-methyl-allyl)-5,6,7,8-tetrahydro-10-thia-5,8-diaza-benzo[a]azulen-9-one;
5-(2-Methyl-allyl)-3-nitro-5,6,7,8-tetrahydro-10-thia-5,8-diaza-benz[a]azulen-9-one;
7-Benzyl-3-methoxy-5-propyl-5,6,7,8-tetrahydro-10-thia-5,8-diaza-benzo[a]azulen-9-one;
5H-[1]Benzothieno[2,3-f][1,2,4]triazolo[4,3-d][1,4]diazepine,6,7-dihydro-7-(2-methyl-2-propenyl)-9-nitro;
5-Isobutyl-7,7-dimethyl-5,6,7,8-tetrahydro-10-thia-5,8-diaza-benz[a]azulen-9-one;
5-(2-Chloro-allyl)-3-methoxy-5,6,7,8-tetrahydro-10-thia-5,8-diaza-benz[a]azulen-9-one;
8-Allyl-3-methoxy-5-(2-methyl-allyl)-5,6,7,8-tetrahydro-10-thia-5,8-diaza-benz[a]azulen-9-one;
8-Benzoyl-5-(2-methyl-allyl)-3-nitro-5,6,7,8-tetrahydro-10-oxa-5,8-diaza-benz[a]azulen-9-one;
5-Allyl-3-fluoro-5,6,7,8-tetrahydro-10-thia-5,8-diaza-benz[a]azulen-9-one;
3-Fluoro-5-prop-2-ynyl-5,6,7,8-tetrahydro-10-thia-5,8-diaza-benz[a]azulen-9-one;
5-Isobutyl-3-methanesulfonyl-5,6,7,8-tetrahydro-10-thia-5,8-diaza-benz[a]azulen-9-one;
5-Acetyl-3-methoxy-5,6,7,8-tetrahydro-10-thia-5,8-diaza-benzo [a]azulen-9-one;
5-Benzyl-3-methoxy-5,6,7,8-tetrahydro-10-thia-5,8-diaza-benzo[a]azulen-9-one; and
3-Chloro-5-propyl-4b,5,6,7,8,9a-hexahydro-10-oxa-5,8-diaza-benzo[a]azulen-9-one.

26. A compound with improved therapeutic index and improved metabolic stability selected from:
8,9,10,11-Tetrahydro-3,8,11-triaza-cyclohepta[a]naphthalen-7-one;
11-(3-Methoxy-benzyl)-8,9,10,11-tetrahydro-3,8,11-triaza-cyclohepta[a]naphthalen-7-one; and
11-Ethyl-8,9,10,11-tetrahydro-3,8,11-triaza-cyclohepta[a]naphthalen-7-one.

27. A method for treating and/or preventing herpes viral infections which comprises administering to a mammal in need of said treatment and/or prevention a compound according to claim 26.

28. A pharmaceutical composition comprising an amount of a compound of claim 1 effective to treat and/or prevent herpes viral infection in a mammal and a pharmaceutically acceptable carrier according to claim 26.

29. A pharmaceutical composition comprising administering a compound according to claim 1 in combination with another compound having improved viral suppression.

30. A pharmaceutical composition according to claim 1 where a compound of claim 1 is in combination with acyclovir.

31. A compound according to claim 1 exhibiting improved metabolic stability, therapeutic index and potency, or a pharmaceutically acceptable salt thereof.

32. A method for treating and/or preventing human herpes viruses 6, 7, and 8 which comprises administering to a mammal in need of said treatment and/or prevention a compound according to claim 1.
